(19) United States
(12) Patent Application Publication
(10) Pub. No.: US 2025/0197206 A1
(43) Pub. Date: Jun. 19, 2025

(54) RECOVERY OF CARBON DIOXIDE FROM METHANE COMBUSTION AND ETHYLENE OXIDE PRODUCTION TO PRODUCE RECYCLE CONTENT SYNGAS

(71) Applicant: Eastman Chemical Company, Kingsport, TN (US)

(72) Inventors: Michael Gary Polasek, Longview, TX (US); David Eugene Slivensky, Tatum, TX (US); Daryl Bitting, Longview, TX (US); Xianchun Wu, Longview, TX (US)

(73) Assignee: Eastman Chemical Company, Kingsport, TN (US)

(21) Appl. No.: 18/845,519

(22) PCT Filed: Mar. 15, 2023
(86) PCT No.: PCT/US2023/064382

§ 371 Date: Sep. 10, 2024

Related U.S. Application Data
(60) Provisional application No. 63/269,474, filed on Mar. 17, 2022.

(51) Int. Cl. C01B 3/06; C07D 301/08; C10G 1/10

(52) U.S. Cl. CPC C01B 3/06; C07D 301/08; C10G 1/10; C01B 2203/025; C01B 2203/062; C01B 2203/1276; C10G 2300/1003; C10G 2300/4006; C10G 2300/4012; C10G 2300/4043

(57) ABSTRACT

It has been discovered that recycle content CO₂ streams produced in a chemical recycling facility involving waste plastic pyrolysis can be converted into recycle content syngas (r-syngas), which can be used for various applications. More particularly, recycle content CO₂ streams generated from a pyrolysis facility, a cracking facility, and/or an ethylene oxide facility may be recovered and converted into r-syngas. Moreover, recycle content methane, produced directly or indirectly from waste plastics, may also be used to produce flue gas streams, which provide additional recycle content CO₂ streams to produce additional recycle content syngas. Thus, methods for producing a useful recycle content product (i.e., r-syngas) from conventional waste streams that are typically exhausted (i.e., CO₂ streams) are provided herein.

### BACKGROUND

Waste plastic pyrolysis plays a part in a variety of chemical recycling technologies. The pyrolysis of waste plastic produces heavy components (e.g., waxes, tar, and char), as well as recycle content pyrolysis oil (r-pyoil) and recycle content pyrolysis gas (r-pygas). When the pyrolysis facility is located near another processing facility, such as a cracker facility, it is desirable to send as much of the r-pyoil and r-pygas as possible to the downstream processing facility to be used as a feedstock in forming other recycle content products (e.g., olefins, paraffins, etc.).

Despite producing recycle content products, various waste materials, such as waste gases, can be produced during these processes that negatively impact the environment when disposed of. Thus, from an environmental standpoint, it is desirable to recycle as much of these waste materials as possible. However, certain waste streams, such as carbon dioxide gas, still exist that are themselves not economically feasible to recover or recycle, resulting in additional waste streams that must be disposed of or otherwise handled. Consequently, this can negatively affect the carbon footprint of a chemical recycling facility.

While some waste materials are relatively easy and inexpensive to recycle, other waste materials require significant and expensive processing in order to be reused, particularly if the material is a gas that is difficult to isolate, such as carbon dioxide. As related to a gas, there is a lack of infrastructure to segregate and distribute a dedicated portion of a gas made exclusively from a recycle content feedstock since the gas infrastructure is continuously fluid and often commingles gas streams from a variety of sources.

Thus, there is a need for one or more processes for recycling waste materials from waste plastic pyrolysis, such as carbon dioxide streams, into more useful and desirable products.

### SUMMARY

In one aspect, the present technology concerns a chemical recycling process. Generally, the process comprises: (a) oxidizing at least a portion of a recycle content ethylene in an oxidation reactor to form a recycle content ethylene oxide stream and a recycle content CO₂ stream; (b) feeding at least a portion of the recycle content CO₂ stream to a POX gasifier; and (c) gasifying at least a portion of the recycle content CO₂ stream in the POX gasifier to thereby form a recycle content syngas.

In one aspect, the present technology concerns a chemical recycling process. Generally, the process comprises: (a) generating a CO₂ stream by—i) oxidizing ethylene to form ethylene oxide and CO₂, and ii) combusting methane to form flue gas comprising CO₂; and (b) gasifying at least a portion of the generated CO₂ in a POX gasifier to thereby form a syngas.

### BRIEF DESCRIPTION OF THE DRAWINGS

FIG. 1 is a block flow diagram illustrating the main steps of a process and facility for chemically recycling waste plastic, forming recycle content carbon dioxide from one or more pyrolysis products, and then forming a recycle content syngas according to embodiments of the present technology;

FIG. 2 is a block flow diagram of an ethylene oxide facility according to embodiments of the present technology;

FIG. 3 is a block flow diagram illustrating the formation of recycle content syngas from flue gas streams according to embodiments of the present technology; and

FIG. 4 is a block flow diagram illustrating the formation of recycle content syngas from various CO₂ streams according to embodiments of the present technology.

### DETAILED DESCRIPTION

We have discovered that CO₂ streams produced in a chemical recycling facility involving waste plastic pyrolysis can be converted into recycle content syngas (r-syngas), which can be used for various applications. More particularly, we have discovered that CO₂ streams generated from a pyrolysis facility, a cracking facility, and/or an ethylene oxide facility may be recovered and converted into r-syngas. Thus, we have discovered methods for producing a useful recycle content product (i.e., r-syngas) from conventional waste streams that are typically exhausted (i.e., CO₂ streams).

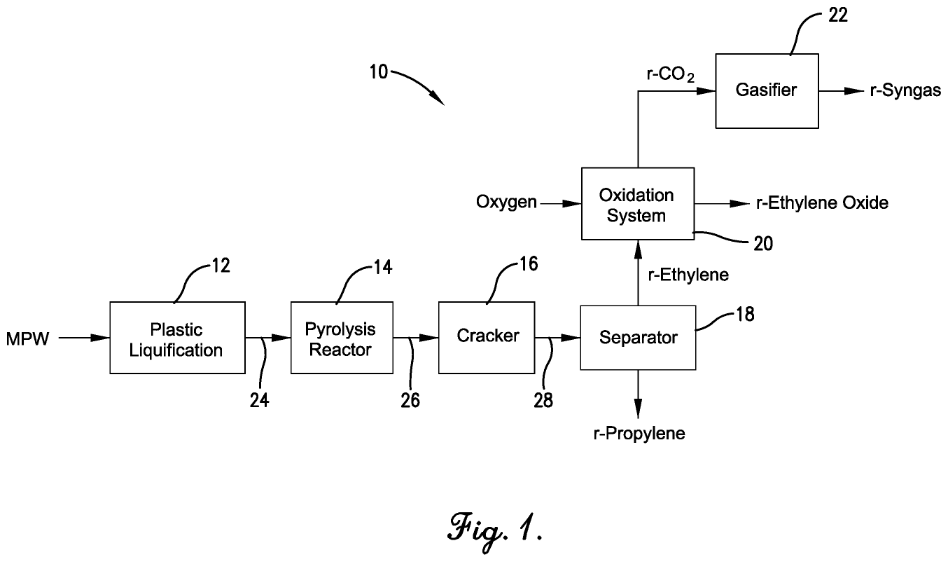

FIG. 1 depicts an exemplary chemical recycling facility 10 comprising a pyrolysis facility (e.g., the plastic liquification system 12 and the pyrolysis reactor 14), a cracking facility (e.g., the cracker furnace 16 and the separator 18), an ethylene oxide facility (e.g., the oxidation system 20), and a molecular reforming facility (e.g., the gasifier 22). As depicted in FIG. 1, waste plastic may be pyrolyzed to produce a pyrolysis effluent, which may then be converted indirectly into various products downstream, including a CO₂ stream. Subsequently, as shown in FIG. 1, at least a portion of this CO₂ stream may be converted in a molecular reforming facility to form a recycle content syngas. It should be understood that FIG. 1 depicts one exemplary embodiment of the present technology. Certain features depicted in FIG. 1 may be omitted and/or additional features described elsewhere herein may be added to the system depicted in FIG. 1. The various process steps are described below in greater detail.

### Overall Chemical Recycling Facility

In an embodiment or in combination with any embodiment mentioned herein, the chemical recycling facility 10 may be a commercial-scale facility capable of processing significant volumes of mixed plastic waste. As used herein, the term “commercial scale facility” refers to a facility having an average annual feed rate of at least 500 pounds per hour, averaged over one year.

Turning now to FIG. 1, the main steps of a process for chemically recycling waste plastic in a chemical recycling facility 10 are shown. Chemical recycling processes and facilities as described herein may be used to convert waste plastic to recycle content products or chemical intermediates used to form a variety of end use materials. The waste plastic fed to the chemical recycling facility/process can be mixed plastic waste (MPW), pre-sorted waste plastic, and/or pre-processed waste plastic. As shown in FIG. 1, the waste plastic feed stream may be derived from a waste plastic source, which may include a waste plastic preprocessing facility.

In an embodiment or in combination with any embodiment mentioned herein, the chemical recycling facility 10 may be a commercial-scale facility capable of processing significant volumes of mixed plastic waste. As used herein, the term “commercial scale facility” refers to a facility having an average annual feed rate of at least 500 pounds per hour, averaged over one year.

In an embodiment or in combination with any embodiment mentioned herein, two or more of the facilities shown in FIG. 1, such as the pyrolysis facility (e.g., the plastic liquification system 12 and the pyrolysis reactor 14), the cracking facility (e.g., the cracker furnace 16 and the separator 18), the ethylene oxide facility (e.g., the oxidation system 20), and/or the molecular reforming facility (e.g., the gasifier 22), may be co-located with one another. As used herein, the term “co-located” refers to facilities in which at least a portion of the process streams and/or supporting equipment or services are shared between the two facilities. When two or more of the facilities shown in FIG. 1 are co-located, the facilities may meet at least one of the following criteria (i) through (v): (i) the facilities share at least one non-residential utility service; (ii) the facilities share at least one service group; (iii) the facilities are owned and/or operated by parties that share at least one property boundary; (iv) the facilities are connected by at least one conduit configured to carry at least one process material (e.g., solid, liquid and/or gas fed to, used by, or generated in a facility) from one facility to another; and (v) the facilities are within 40, within 35, within 30, within 20, within 15, within 12, within 10, within 8, within 5, within 2, or within 1 mile of one another, measured from their geographical center. At least one, at least two, at least three, at least four, or all of the above statements (i) through (v) may be true.

Regarding (i), examples of suitable utility services include, but are not limited to, steam systems (co-generation and distribution systems), cooling water systems, heat transfer fluid systems, plant or instrument air systems, nitrogen systems, hydrogen systems, non-residential electrical generation and distribution, including distribution above 8000V, non-residential wastewater/sewer systems, storage facilities, transport lines, flare systems, and combinations thereof.

Regarding (ii), examples of service groups and facilities include, but are not limited to, emergency services personnel (fire and/or medical), a third-party vendor, a state or local government oversight group, and combinations thereof. Government oversight groups can include, for example, regulatory or environmental agencies, as well as municipal and taxation agencies at the city, county, and state level.

Regarding (iii), the boundary may be, for example, a fence line, a property line, a gate, or common boundaries with at least one boundary of a third-party owned land or facility.

Regarding (iv), the conduit may be a fluid conduit that carries a gas, a liquid, a solid/liquid mixture (e.g., slurry), a solid/gas mixture (e.g., pneumatic conveyance), a solid/liquid/gas mixture, or a solid (e.g., belt conveyance). In some cases, two units may share one or more conduits selected from the above list.

Turning again to FIG. 1, a stream of waste plastic, which can be mixed plastic waste (MPW), may be introduced into the chemical recycling facility 10 from the waste plastic source. As used herein, the terms “waste plastic” and “plastic waste” refer to used, scrap, and/or discarded plastic materials, such as plastic materials typically sent to a landfill. The waste plastic stream fed to the chemical recycling facility 10 may include unprocessed or partially processed waste plastic. As used herein, the term “unprocessed waste plastic” means waste plastic that has not be subjected to any automated or mechanized sorting, washing, or comminuting. Examples of unprocessed waste plastic include waste plastic collected from household curbside plastic recycling bins or shared community plastic recycling containers. Partially processed waste plastics may originate from, for example, municipal recycling facilities (MRFs) or reclaimers. In certain embodiments, the waste plastic may comprise at least one of post-industrial (or pre-consumer) plastic and/or post-consumer plastic.

In an embodiment or in combination with any embodiment mentioned herein, the mixed waste plastic (MPW) includes at least two distinct types of plastic.

In an embodiment or in combination with any embodiment mentioned herein, all or a portion of the MPW in the waste plastic stream can originate from a municipal recycling facility (MRF).

In an embodiment or in combination with any embodiment mentioned herein, all or a portion of the MPW in the waste plastic stream can originate from a reclaimer facility.

Examples of suitable waste plastics can include, but are not limited to, polyolefins (PO), aromatic and aliphatic polyesters, polyvinyl chloride (PVC), polystyrene, cellulose esters, polytetrafluoroethylene, acrylobutadienestyrene (ABS), cellulosics, epoxides, polyamides, phenolic resins, polyacetal, polycarbonates, polyphenylene-based alloys, poly(methyl methacrylate), styrene-containing polymers, polyurethane, vinyl-based polymers, styrene acrylonitrile, and urea-containing polymers and melamines.

Examples of specific polyolefins may include linear low-density polyethylene (LLDPE), low density polyethylene (LDPE), polymethylpentene, polybutene-1, high density polyethylene (HDPE), atactic polypropylene, isotactic polypropylene, syndiotactic polypropylene, crosslinked polyethylene, amorphous polyolefins, and the copolymers of any one of the aforementioned polyolefins.

Examples of polyesters can include those having repeating aromatic or cyclic units such as those containing a repeating terephthalate, isophthalate, or naphthalate units such as PET, modified PET, and PEN, or those containing repeating furanate repeating units. As used herein, “PET” or “polyethylene terephthalate” refers to a homopolymer of polyethylene terephthalate, or to a polyethylene terephthalate modified with one or more acid and/or glycol modifiers and/or containing residues or moieties of other than ethylene glycol and terephthalic acid, such as isophthalic acid, 1,4-cyclohexanedicarboxylic acid, diethylene glycol, 2,2,4,4-tetramethyl-1,3-cyclobutanediol (TMCD), cyclohexanedimethanol (CHDM), propylene glycol, isosorbide, 1,4-butanediol, 1,3-propane diol, and/or neopentyl glycol (NPG).

In an embodiment or in combination with any embodiment mentioned herein, the waste plastic stream comprises at least 20, at least 25, at least 30, at least 35, at least 40, at least 45, at least 50, at least 55, at least 60, at least 65, at least 70, at least 75, at least 80, at least 85, at least 90, at least 95, or at least 99 weight percent of one or more polyolefins, based on the total weight of the stream. Alternatively, or in addition, the waste plastic stream comprises not more than 99.9, not more than 99, not more than 97, not more than 92, not more than 90, not more than 85, not more than 80, not more than 75, not more than 70, not more than 65, not more than 60, not more than 55, not more than 50, not more than 45, not more than 40, not more than 35, not more than 30, not more than 25, not more than 20, not more than 15, not more than 10, or not more than 5 weight percent of one or more polyolefins, based on the total weight of the stream.

In one embodiment or in combination with any of the mentioned embodiments, the waste plastic stream comprises not more than 20, not more than 15, not more than 12, not more than 10, not more than 8, not more than 6, not more than 5, not more than 4, not more than 3, not more than 2, or not more than 1 weight percent of polyesters, based on the total weight of the stream.

In one embodiment or in combination with any of the mentioned embodiments, the waste plastic stream comprises not more than 20, not more than 15, not more than 12, not more than 10, not more than 8, not more than 6, not more than 5, not more than 4, not more than 3, not more than 2, or not more than 1 weight percent of biowaste materials, based on the total weight of the stream. As used herein, the term “biowaste” refers to material derived from living organisms or of organic origin. Exemplary biowaste materials include, but are not limited to, cotton, wood, saw dust, food scraps, animals and animal parts, plants and plant parts, and manure.

In an embodiment or in combination with any embodiment mentioned herein, the waste plastic stream can include not more than 10, not more than 5, not more than 4, not more than 3, not more than 2, not more than 1, not more than 0.75, or not more than 0.5 weight percent of polyvinyl chloride (PVC), based on the total weight of the stream.

The general configuration and operation of each of the facilities that may be present in the chemical recycling facility 10 shown in FIG. 1 will now be described in further detail below, beginning with the optional preprocessing facility of the waste plastic source.

### Optional Plastic Preprocessing

As shown in FIG. 1, unprocessed, partially processed, and/or processed waste plastic, such as mixed plastic waste (MPW), may first be introduced into the chemical recycling facility 10 via the waste plastic stream from a waste plastic source. The waste plastic source may also include an optional preprocessing facility that can prepare the waste plastic feedstock for the downstream recycling processes. While in the optional preprocessing facility, the waste plastic feedstock may undergo one or more preprocessing steps to prepare it for chemical recycling. As used herein, the term “preprocessing facility” refers to a facility that includes all equipment, lines, and controls necessary to carry out the preprocessing of waste plastic. Preprocessing facilities as described herein may employ any suitable method for carrying out the preparation of waste plastic for chemical recycling using one or more of following steps, which are described in further detail below. Alternatively, in certain embodiments, the waste plastic source does not contain a preprocessing facility and the waste plastic stream is not subjected to any preprocessing before any of the downstream chemical recycling steps described herein.

In an embodiment or in combination with any embodiment mentioned herein, the preprocessing facility of the waste plastic source may include at least one separation step or zone. The separation step or zone may be configured to separate the waste plastic stream into two or more streams enriched in certain types of plastics. Such separation is particularly advantageous when the waste plastic fed to the chemical recycling facility 10 is MPW.

Any suitable type of separation device, system, or facility may be employed to separate the waste plastic into two or more streams enriched in certain types of plastics such as, for example, a PET-enriched stream and a PO-enriched stream. Examples of suitable types of separation include mechanical separation and density separation, which may include sink-float separation and/or centrifugal density separation. As used herein, the term “sink-float separation” refers to a density separation process where the separation of materials is primarily caused by floating or sinking in a selected liquid medium, while the term “centrifugal density separation” refers to a density separation process where the separation of materials is primarily caused by centrifugal forces.

Referring again to FIG. 1, the waste plastic stream may be introduced into one or more downstream processing facilities (or undergo one or more downstream processing steps) within the chemical recycling facility 10. In an embodiment or in combination with any embodiment mentioned herein, at least a portion of the waste plastic stream may be directly or indirectly introduced into a plastic liquification system 12 within the waste plastic source or outside of it. Additional details of each step, as well as the general integration of each of these steps or facilities with one or more of the others according to one or more embodiments of the present technology are discussed in further detail below.

### Liquification/Dehalogenation

As shown in FIG. 1, the waste plastic stream may be introduced into a plastic liquification system 12 prior to being introduced into the pyrolysis reactor 14. As used herein, the term “liquification” system refers to a chemical processing zone or step in which at least a portion of the incoming plastic is liquefied. The step of liquefying plastic in FIG. 1 can include chemical liquification, physical liquification, or combinations thereof. Exemplary methods of liquefying the plastic introduced in the liquification system 12 can include: (i) heating/melting; (ii) dissolving in a solvent; (iii) depolymerizing; (iv) plasticizing; and combinations thereof. Additionally, one or more of options (i) through (iv) may also be accompanied by the addition of a blending or liquification agent to help facilitate the liquification (reduction of viscosity) of the polymer material. As such, a variety of rheology modification agents (e.g., solvents, depolymerization agents, plasticizers, and blending agents) can be used the enhance the flow and/or dispersibility of the liquified waste plastic.

When added to the liquification system 12, at least 50, at least 55, at least 60, at least 65, at least 70, at least 75, at least 80, at least 85, at least 90, at least 95, or at least 99 weight percent of the plastic (usually waste plastic) originally present in the waste plastic stream undergoes a reduction in viscosity. In some cases, the reduction in viscosity can be facilitated by heating (e.g., addition of steam directly or indirectly contacting the plastic), while, in other cases, it can be facilitated by combining the plastic with a solvent capable of dissolving it. Examples of suitable solvents can include, but are not limited to, alcohols such as methanol or ethanol, glycols such as ethylene glycol, diethylene glycol, triethylene glycol, neopentyl glycol, cyclohexanedimethanol, glycerin, pyrolysis oil, motor oil, and water. This dissolution solvent can be added directly to the liquification vessel in the liquification system 12, or it can be previously combined with one or more streams fed to the liquification system 12, including the waste plastic stream.

In an embodiment or in combination with any embodiment mentioned herein, the dissolution solvent can comprise a stream withdrawn from one or more other facilities within the chemical recycling facility 10. For example, the solvent can comprise a stream withdrawn from the pyrolysis reactor 14. In certain embodiments, the dissolution solvent can be or comprise pyrolysis oil.

In some cases, the waste plastic can be depolymerized such that, for example, the number average chain length of the plastic is reduced by contact with a depolymerization agent. In an embodiment or in combination with any embodiment mentioned herein, at least one of the previously-listed solvents may be used as a depolymerization agent, while, in one or more other embodiments, the depolymerization agent can include an organic acid (e.g., acetic acid, citric acid, butyric acid, formic acid, lactic acid, oleic acid, oxalic, stearic acid, tartaric acid, and/or uric acid) or inorganic acid such as sulfuric acid (for polyolefins). The depolymerization agent may reduce the melting point and/or viscosity of the polymer by reducing its number average chain length.

Alternatively, or additionally, a plasticizer can be used in the liquification system 12 to reduce the viscosity of the plastic. Plasticizers for polyethylene include, for example, dioctyl phthalate, dioctyl terephthalate, glyceryl tribenzoate, polyethylene glycol having molecular weight of up to 8,000 Daltons, sunflower oil, paraffin wax having molecular weight from 400 to 1,000 Daltons, paraffinic oil, mineral oil, glycerin, EPDM, and EVA. Plasticizers for polypropylene include, for example, dioctyl sebacate, paraffinic oil, isooctyl tallate, plasticizing oil (Drakeol 34), naphthenic and aromatic processing oils, and glycerin. Plasticizers for polyesters include, for example, polyalkylene ethers (e.g., polyethylene glycol, polytetramethylene glycol, polypropylene glycol or their mixtures) having molecular weight in the range from 400 to 1500 Daltons, glyceryl monostearate, octyl epoxy soyate, epoxidized soybean oil, epoxy tallate, epoxidized linseed oil, polyhydroxyalkanoate, glycols (e.g., ethylene glycol, pentamethylene glycol, hexamethylene glycol, etc.), phthalates, terephthalates, trimellitate, and polyethylene glycol di-(2-ethylhexoate). When used, the plasticizer may be present in an amount of at least 0.1, at least 0.5, at least 1, at least 2, or at least 5 weight percent and/or not more than 10, not more than 8, not more than 5, not more than 3, not more than 2, or not more than 1 weight percent, based on the total weight of the waste plastic stream, or it can be in a range of from 0.1 to 10 weight percent, 0.5 to 8 weight percent, or 1 to 5 weight percent, based on the total weight of the waste plastic stream.

Further, one or more of the methods of liquefying the waste plastic stream can also include adding at least one blending agent to the plastic stream before, during, or after the liquification process in the liquification system 12. Such blending agents may include for example, emulsifiers and/or surfactants, and may serve to more fully blend the liquified plastic into a single phase, particularly when differences in densities between the plastic components of a mixed plastic stream result in multiple liquid or semi-liquid phases. When used, the blending agent may be present in an amount of at least 0.1, at least 0.5, at least 1, at least 2, or at least 5 weight percent and/or not more than 10, not more than 8, not more than 5, not more than 3, not more than 2, or not more than 1 weight percent, based on the total weight of the waste plastic stream, or it can be in a range of from 0.1 to 10 weight percent, 0.5 to 8 weight percent, or 1 to 5 weight percent, based on the total weight of the waste plastic stream.

In an embodiment or in combination with any embodiment mentioned herein, a portion of the pyrolysis oil stream from the pyrolysis reactor 14 can be combined with the waste plastic stream to form a liquified plastic. Generally, in such embodiments, all or a portion of the pyrolysis oil stream may be combined with the waste plastic stream prior to introduction into the liquification system 12, or after the waste plastic stream enters the liquification vessel within the liquification system 12.

In an embodiment or in combination with any embodiment mentioned herein, the liquified (or reduced viscosity) plastic stream withdrawn from the liquification system 12 can include at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 weight percent and/or not more than 95, not more than 90, not more than 85, not more than 80, not more than 75, not more than 70, not more than 65, not more than 60, not more than 55, not more than 50, not more than 45, not more than 40, not more than 35, not more than 30, not more than 25, not more than 20, not more than 15, not more than 10, not more than 5, not more than 2, or not more than 1 weight percent of one or more polyolefins, based on the total weight of the stream, or the amount of polyolefins can be in the range of from 1 to 99 weight percent, 5 to 90 weight percent, or 10 to 85 weight percent, based on the total weight of the stream.

In an embodiment or in combination with any embodiment mentioned herein, the liquified plastic stream 24 exiting the plastic liquification system 12 can have a viscosity of less than 3,000, less than 2,500, less than 2,000, less than 1,500, less than 1,000, less than 800, less than 750, less than 700, less than 650, less than 600, less than 550, less than 500, less than 450, less than 400, less than 350, less than 300, less than 250, less than 150, less than 100, less than 75, less than 50, less than 25, less than 10, less than 5, or less than 1 poise, as measured using a Brookfield R/S rheometer with a V80-40 vane spindle operating at a shear rate of 10 rad/s and a temperature of 350° C.

In an embodiment or in combination with any embodiment mentioned herein, the plastic liquification system 12 may comprise at least one liquification vessel to facilitate the liquefying of the waste plastics. In various embodiments, the liquification vessel can include at least one melt tank and/or at least one extruder to facilitate the plastic liquification. Additionally, in certain embodiments, the liquification system 12 may also contain at least one stripping column and at least one disengagement vessel to facilitate the removal of halogenated compounds that may be formed in the liquification vessel.

In an embodiment or in combination with any embodiment mentioned herein, the melt tank can include one or more continuously stirred tanks. When one or more rheology modification agents (e.g., solvents, depolymerization agents, plasticizers, and blending agents) are used in the liquification system 12, such rheology modification agents can be added to and/or mixed with the waste plastic stream in or prior to introduction into the melt tank.

In an embodiment or in combination with any embodiment mentioned herein, the liquification vessel, such as the melt tank and/or the extruder, may receive the waste plastic feed stream and heat the waste plastic via heating mechanisms in the melt tank and/or via the extrusion process in the extruder.

In an embodiment or in combination with any embodiment mentioned herein, the interior space of the liquification vessel, where the plastic is heated, is maintained at a temperature of at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, or at least 400° C. Additionally, or in the alternative, the interior space of the liquification vessel may be maintained at a temperature of not more than 500, not more than 475, not more than 450, not more than 425, not more than 400, not more than 390, not more than 380, not more than 370, not more than 365, not more than 360, not more than 355, not more than 350, or not more than 345° C. Generally, in one or more embodiments, the interior space of the liquification vessel may be maintained at a temperature ranging from 200 to 500° C., 240 to 425° C., 280 to 380° C., or 320 to 350° C.

In an embodiment or in combination with any embodiment mentioned herein, the liquification vessel, such as the melt tank and/or the extruder, may be at least partially heated by a combustion system comprising a plurality a burners that combust a combustion fuel and a combustion air. Furthermore, this combustion system may produce a flue gas stream that can be removed from the liquification system 12. The combustion fuel may comprise a conventional fossil fuel and/or a recycle content fuel, such as recycle content alkanes (e.g., r-methane) and/or recycle content hydrogen derived from the chemical recycling facility.

In an embodiment or in combination with any embodiment mentioned herein, the liquification system 12 may optionally contain equipment for removing halogens from the waste plastic stream. When the waste plastic is heated in the liquification system 12, halogen enriched gases can evolve. By disengaging the evolved halogen-enriched gasses from the liquified plastics, the concentration of halogens in the liquified plastic stream 24 can be reduced.

In an embodiment or in combination with any embodiment mentioned herein, dehalogenation can be promoted by sparging a stripping gas (e.g., steam) into the liquified plastics in the melt tank.

In an embodiment or in combination with any embodiment mentioned herein, the liquified plastic stream 24 exiting the liquification system 12 can have a halogen content of less than 500, less than 400, less than 300, less than 200, less than 100, less than 50, less than 10, less than 5, less than 2, less than 1, less than 0.5, or less than 0.1 ppmw.

In an embodiment or in combination with any embodiment mentioned herein, the liquefied waste plastic stream exiting the plastic liquification system 12 may have a temperature of at least 200, at least 225, at least 250, at least 275, at least 300, at least 310, at least 320, at least 330, or at least 340° C. and/or less than 450, less than 425, less than 400, less than 375, or less than 350° C.

As shown in FIG. 1 and described below in greater detail, at least a portion of the liquified plastic stream 24 may be introduced into a downstream pyrolysis reactor 14 at a pyrolysis facility to produce a pyrolysis effluent, including a pyrolysis oil and a pyrolysis gas.

### Pyrolysis

As shown in FIG. 1, the chemical recycling facility 10 may comprise a pyrolysis reactor 14. As used herein, the term “pyrolysis” refers to thermal decomposition of a feedstock of a biomass and/or a plastic material in solid or liquid form at elevated temperatures in an inert (i.e., substantially molecular oxygen free) atmosphere. A “pyrolysis facility” is a facility that includes all equipment, lines, and controls necessary to carry out pyrolysis of waste plastic and feedstocks derived therefrom. In certain embodiments, the pyrolysis facility can comprise the pyrolysis reactor 14 and, optionally, the plastic liquification system 12 and/or the waste plastic source.

As depicted in FIG. 1, the liquified plastic stream 24 may be introduced into a downstream pyrolysis reactor 14 at a pyrolysis facility so as to produce a pyrolysis effluent stream comprising a pyrolysis oil, a pyrolysis gas, and a pyrolysis residue.

In an embodiment or in combination with any embodiment mentioned herein, the liquified plastic stream 24 to the pyrolysis facility may be a PO-enriched stream of waste plastic. The liquified plastic stream 24 introduced into the pyrolysis reactor 14 can be in the form of liquified plastic (e.g., liquified, melted, plasticized, depolymerized, or combinations thereof), plastic pellets or particulates, or a slurry thereof.

In general, the pyrolysis facility may include the plastic liquification system 12, the pyrolysis reactor 14, and a separation system (not shown in FIG. 1) for the pyrolysis effluent, which can separate the pyrolysis effluent into a pyrolysis gas stream, a pyrolysis oil stream, and/or a pyrolysis residue stream.

While in the pyrolysis reactor 14, at least a portion of the feed may be subjected to a pyrolysis reaction that produces a pyrolysis effluent 26 comprising a pyrolysis oil, a pyrolysis gas, and a pyrolysis residue. Generally, the pyrolysis effluent stream 26 exiting the pyrolysis reactor 14 can be in the form of pyrolysis vapors that comprise the pyrolysis gas and uncondensed pyrolysis oil. As used herein, “pyrolysis vapor” refers to the uncondensed pyrolysis effluent that comprises the majority of the pyrolysis oil and the pyrolysis gas present in the pyrolysis effluent.

Pyrolysis is a process that involves the chemical and thermal decomposition of the introduced feed. Although all pyrolysis processes may be generally characterized by a reaction environment that is substantially free of oxygen, pyrolysis processes may be further defined, for example, by the pyrolysis reaction temperature within the reactor, the residence time in the pyrolysis reactor 14, the reactor type, the pressure within the pyrolysis reactor 14, and the presence or absence of pyrolysis catalysts.

In an embodiment or in combination with any embodiment mentioned herein, the pyrolysis reactor 14 can be, for example, a film reactor, a screw extruder, a tubular reactor, a tank, a stirred tank reactor, a riser reactor, a fixed bed reactor, a fluidized bed reactor, a rotary kiln, a vacuum reactor, a microwave reactor, or an autoclave.

In an embodiment or in combination with any embodiment mentioned herein, a lift gas and/or a feed gas may be used to introduce the feedstock into the pyrolysis reactor 14 and/or facilitate various reactions within the pyrolysis reactor 14. For instance, the lift gas and/or the feed gas may comprise, consist essentially of, or consist of nitrogen, carbon dioxide, and/or steam. The lift gas and/or feed gas may be added with the waste plastic stream prior to introduction into the pyrolysis reactor 14 and/or may be added directly to the pyrolysis reactor 14. The lift gas and/or feed gas can include steam and/or a reducing gas such as hydrogen, carbon monoxide, and combinations thereof.

Furthermore, the temperature in the pyrolysis reactor 14 can be adjusted so as to facilitate the production of certain end products. In an embodiment or in combination with any embodiment mentioned herein, the pyrolysis temperature in the pyrolysis reactor 14 can range from 325 to 1,100° C., 350 to 900° C., 350 to 700° C., 350 to 550° C., 350 to 475° C., 425 to 1,100° C., 425 to 800° C., 500 to 1,100° C., 500 to 800° C., 600 to 1,100° C., 600 to 800° C., 650 to 1,000° C., 700 to 1,000° C., or 650 to 800° C. Generally, in certain embodiments, the pyrolysis temperature in the pyrolysis reactor 14 can be greater than 650° C.

In an embodiment or in combination with any embodiment mentioned herein, the residence times of the feedstocks within the pyrolysis reactor 14 can be at least 0.1, at least 0.2, at least 0.3, at least 0.5, at least 1, at least 1.2, at least 1.3, at least 2, at least 3, or at least 4 seconds. Alternatively, the residence times of the feedstocks within the pyrolysis reactor 14 can be at least 1, at least 2, at least 3, at least 4, at least 5, at least 6, at least 7, at least 8, at least 9, at least 10, at least 20, at least 30, at least 45, at least 60, at least 75, or at least 90 minutes. Additionally, or alternatively, the residence times of the feedstocks within the pyrolysis reactor 14 can be less than 6, less than 5, less than 4, less than 3, less than 2, less than 1, or less than 0.5 hours. Furthermore, the residence times of the feedstocks within the pyrolysis reactor 14 can be less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or less than 1 seconds. More particularly, the residence times of the feedstocks within the pyrolysis reactor 14 can range from 0.1 to 10 seconds, 0.5 to 10 seconds, 30 minutes to 4 hours, or 30 minutes to 3 hours, or 1 hour to 3 hours, or 1 hour to 2 hours.

In an embodiment or in combination with any embodiment mentioned herein, the pressure within the pyrolysis reactor 14 can be maintained at atmospheric pressure or within the range of 0.1 to 100 bar, or 0.1 to 60 bar, or 0.1 to 30 bar, or 0.1 to 10 bar, 0.2 to 1.5 bar, or 0.3 to 1.1 bar. As used herein, the term “bar” refers to gauge pressure, unless otherwise noted.

In an embodiment or in combination with any embodiment mentioned herein, a pyrolysis catalyst may be introduced into the liquified plastic stream 24 prior to introduction into the pyrolysis reactor 14 and/or introduced directly into the pyrolysis reactor 14. The catalyst can be homogenous or heterogeneous and may include, for example, certain types of zeolites and other mesostructured catalysts. In some embodiments, the pyrolysis reaction may not be catalyzed (e.g., carried out in the absence of a pyrolysis catalyst), but may include a non-catalytic, heat-retaining inert additive, such as sand, in the reactor in order to facilitate the heat transfer. Such catalyst-free pyrolysis processes may be referred to as “thermal pyrolysis.”

In an embodiment or in combination with any embodiment mentioned herein, the pyrolysis reactor 14 may be at least partially heated by a combustion system comprising a plurality a burners that combust a combustion fuel and a combustion air. Furthermore, this combustion system may produce a flue gas stream that can be removed from the pyrolysis reactor 14. The combustion fuel may comprise a conventional fossil fuel and/or a recycle content fuel, such as recycle content alkanes (e.g., r-methane) and/or recycle content hydrogen derived from the chemical recycling facility.

After exiting the pyrolysis reactor 14, the pyrolysis effluent 26 may be separated into a recycle content pyrolysis oil stream, a recycle content pyrolysis residue stream, and a recycle content pyrolysis gas stream in a separation system. Although not depicted in FIG. 1, this separation system can include various types of equipment including, but not limited to a filter system, a multistage separator, a condensation zone, a distillation column, and/or a quench tower. While in the separation system, the pyrolysis effluent 26, such as the pyrolysis vapors, may be cooled to condense the pyrolysis oil fraction originally present in the pyrolysis effluent stream.

In an embodiment or in combination with any embodiment mentioned herein, the pyrolysis effluent or pyrolysis vapors from the pyrolysis reactor 14 may comprise at least 1, at least 5, at least 10, at least 15, at least 20, at least 25, at least 30, at least 35, at least 40, at least 45, at least 50, at least 55, at least 60, at least 65, at least 70, or at least 75 weight percent of the pyrolysis oil, based on the total weight of the pyrolysis effluent or pyrolysis vapors. Additionally, or alternatively, the pyrolysis effluent or pyrolysis vapors may comprise not more than 99, not more than 95, not more than 90, not more than 85, not more than 80, not more than 75, not more than 70, not more than 65, not more than 60, not more than 55, not more than 50, not more than 45, not more than 40, not more than 35, not more than 30, or not more than 25 weight percent of the pyrolysis oil, based on the total weight of the pyrolysis effluent or pyrolysis vapors. As discussed above, the pyrolysis oil may be in the form of uncondensed vapors in the pyrolysis effluent upon exiting the heated reactor; however, these vapors may be subsequently condensed into the resulting pyrolysis oil. The pyrolysis effluent or pyrolysis vapors may comprise in the range of 20 to 99 weight percent, 25 to 80 weight percent, 30 to 85 weight percent, 30 to 80 weight percent, 30 to 75 weight percent, 30 to 70 weight percent, or 30 to 65 weight percent of the pyrolysis oil, based on the total weight of the pyrolysis effluent or pyrolysis vapors.

In an embodiment or in combination with any embodiment mentioned herein, the pyrolysis effluent or pyrolysis vapors from the pyrolysis reactor 14 may comprise at least 1, at least 5, at least 10, at least 15, at least 20, at least 25, at least 30, at least 35, at least 40, at least 45, at least 50, at least 55, at least 60, at least 65, at least 70, at least 75, or at least 80 weight percent of the pyrolysis gas, based on the total weight of the pyrolysis effluent or pyrolysis vapors. Additionally, or alternatively, the pyrolysis effluent or pyrolysis vapors may comprise not more than 99, not more than 95, not more than 90, not more than 85, not more than 80, not more than 75, not more than 70, not more than 65, not more than 60, not more than 55, not more than 50, or not more than 45 weight percent of the pyrolysis gas, based on the total weight of the pyrolysis effluent or pyrolysis vapors. The pyrolysis effluent may comprise 1 to 90 weight percent, 10 to 85 weight percent, 15 to 85 weight percent, 20 to 80 weight percent, 25 to 80 weight percent, 30 to 75 weight percent, or 35 to 75 weight percent of the pyrolysis gas, based on the total weight of the stream.

In an embodiment or in combination with any embodiment mentioned herein, the pyrolysis effluent or pyrolysis vapors from the pyrolysis reactor 14 may comprise at least 0.5, at least 1, at least 2, at least 3, at least 4, at least 5, at least 6, at least 7, at least 8, at least 9, or at least 10 weight percent of the pyrolysis residue, based on the total weight of the pyrolysis effluent or pyrolysis vapors. Additionally, or alternatively, the pyrolysis effluent may comprise not more than 60, not more than 50, not more than 40, not more than 30, not more than 25, not more than 20, not more than 15, not more than 10, not more than 9, not more than 8, not more than 7, not more than 6, or not more than 5 weight percent of the pyrolysis residue, based on the total weight of the pyrolysis effluent or pyrolysis vapors. The pyrolysis effluent may comprise in the range of 0.1 to 25 weight percent, 1 to 15 weight percent, 1 to 8 weight percent, or 1 to 5 weight percent of the pyrolysis residue, based on the total weight of the pyrolysis effluent or pyrolysis vapors. This pyrolysis residue may be removed from the pyrolysis reactor 14 (where it may form) and/or separated from the pyrolysis effluent 26 in a downstream separator, such as the condenser.

The resulting pyrolysis oil stream and pyrolysis gas stream may be directly used in various downstream applications based on their formulations. The various characteristics and properties of the pyrolysis oil, pyrolysis gas, and pyrolysis residue are described below. It should be noted that, while all of the following characteristics and properties may be listed separately, it is envisioned that each of the following characteristics and/or properties of the pyrolysis gas, pyrolysis oil, and/or pyrolysis residue are not mutually exclusive and may be combined and present in any combination.

In an embodiment or in combination with any embodiment mentioned herein, the pyrolysis oil may predominantly comprise hydrocarbons having from 4 to 30 carbon atoms per molecule (e.g., C4 to C30 hydrocarbons). As used herein, the term “Cx” or “Cx hydrocarbon,” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4.” The pyrolysis oil may have a C4-C30 hydrocarbon content of at least 55, at least 60, at least 65, at least 70, at least 75, at least 80, at least 85, at least 90, or at least 95 weight percent based on the total weight of the pyrolysis oil stream.

In an embodiment or in combination with any embodiment mentioned herein, the pyrolysis oil can predominantly comprise C5 to C25 hydrocarbons, C5 to C22 hydrocarbons, or C5 to C20 hydrocarbons. For example, the pyrolysis oil may comprise at least 55, at least 60, at least 65, at least 70, at least 75, at least 80, at least 85, at least 90, or at least 95 weight percent of C5 to C25 hydrocarbons, C5 to C22 hydrocarbons, or C5 to C20 hydrocarbons, based on the total weight of the pyrolysis oil stream.

In an embodiment or in combination with any embodiment mentioned herein, the pyrolysis oil may have a mid-boiling point in the range of 75 to 250° C., 90 to 225° C., or 115 to 190° C. as measured according to ASTM D-5399. As used herein, “mid-boiling point” refers to the median boiling point temperature of the pyrolysis oil, where 50 percent by volume of the pyrolysis oil boils above the mid-boiling point and 50 percent by volume boils below the mid-boiling point.

In an embodiment or in combination with any embodiment mentioned herein, the boiling point range of the pyrolysis oil may be such that at least 90 percent of the pyrolysis oil boils off at a temperature of 250° C., of 280° C., of 290° C., of 300° C., or of 310° C., as measured according to ASTM D-5399.

As noted above, the pyrolysis conditions, such as temperature, may be controlled so as to maximize the production of certain hydrocarbons and chemical compounds in the resulting pyrolysis gas and pyrolysis oil.

Turning to the pyrolysis gas, the pyrolysis gas can have a methane content in the range of 1 to 50 weight percent, 5 to 50 weight percent, or 15 to 45 weight percent, based on the total weight of the pyrolysis gas stream.

In an embodiment or in combination with any embodiment mentioned herein, the pyrolysis gas can have a C3 and/or C4 hydrocarbon content (including all hydrocarbons having 3 or 4 carbon atoms per molecule) in the range of 10 to 90 weight percent, 25 to 90 weight percent, or 25 to 80 weight percent, based on the total weight of the pyrolysis gas stream.

In an embodiment or in combination with any embodiment mentioned herein, the pyrolysis gas can have a combined ethylene and propylene content of at least 25, at least 40, at least 50, at least 60, at least 70, or at least 75 weight percent, based on the total weight of the pyrolysis gas stream.

Turning to the pyrolysis residue, in an embodiment or in combination with any embodiment mentioned herein, the pyrolysis residue comprises at least 20, at least 25, at least 30, at least 35, at least 40, at least 45, at least 50, at least 55, at least 60, at least 65, at least 70, at least 75, at least 80, or at least 85 weight percent of C20+ hydrocarbons based on the total weight of the pyrolysis residue. As used herein, “C20+ hydrocarbon” refers to hydrocarbon compounds containing at least 20 total carbons per molecule, and encompasses all olefins, paraffins, and isomers having that number of carbon atoms.

As shown in FIG. 1, at least a portion of the pyrolysis effluent 26, such as the recycle content pyrolysis gas stream, the recycle content pyrolysis oil steam, and/or the recycle content pyrolysis residue stream, may be routed to one or more other chemical processing facilities, including, for example, the cracking facility. In an embodiment or in combination with any embodiment mentioned herein, at least a portion of the pyrolysis effluent 26, such as the recycle content pyrolysis oil stream and/or the recycle content pyrolysis gas stream, may be routed to the cracker furnace 16 of the cracking facility.

### Cracking

As shown in FIG. 1, at least a portion of one or more streams from the pyrolysis facility, including the recycle content pyrolysis oil stream and/or the recycle content pyrolysis gas stream, may be introduced into a cracking facility. As used herein, the term “cracking” refers to breaking down complex organic molecules into simpler molecules by the breaking of carbon-carbon bonds. A “cracking facility” is a facility that includes all equipment, lines, and controls necessary to carry out cracking of a feedstock derived from waste plastic. A cracking facility can include one or more cracker furnaces, a quench system for cooling the cracked products, a compression system, and a downstream separation zone including equipment used to process the effluent of the cracker furnace(s). As used herein, the terms “cracker” and “cracking” are used interchangeably.

As shown in FIG. 1, the cracker facility may include a cracker furnace 16 and a separation zone downstream of the cracker furnace 16 for separating the furnace effluent 28 into various end products, such as a recycle content hydrocarbons (e.g., r-propylene, r-ethylene, and/or r-methane). In an embodiment or in combination with any embodiment mentioned herein, at least a portion of the pyrolysis oil stream and/or the pyrolysis gas stream can be sent to the cracking facility. The pyrolysis oil stream may be introduced into an inlet of the cracker furnace 16, while the pyrolysis gas stream can be introduced into a location upstream or downstream of the furnace. As shown in FIG. 1, the effluent from the cracker furnace 16 may be separated into various recycle content products in the downstream separator 18, such as an r-ethylene stream and a r-propylene stream. When used, the pyrolysis oil stream and/or pyrolysis gas stream may optionally be combined with a stream of cracker feed to form the feed stream to the cracking facility.

In In an embodiment or in combination with any embodiment mentioned herein, at least a portion of the pyrolysis oil stream and/or the pyrolysis gas stream may bypass the cracker furnace 16 and the compression system and be sent directly into the downstream separator 18 in order to recover desired hydrocarbon products therefrom.

In an embodiment or in combination with any embodiment mentioned herein, the cracker feed stream can comprise at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, or at least 95 weight percent of pyrolysis gas, pyrolysis oil, or pyrolysis gas and pyrolysis oil combined, based on the total weight of the stream. Alternatively, or in addition, the cracker feed stream can comprise not more than 95, not more than 90, not more than 85, not more than 80, not more than 75, not more than 70, not more than 65, not more than 60, not more than 55, not more than 50, not more than 45, not more than 40, not more than 35, not more than 30, not more than 25, or not more than 20 weight percent of pyrolysis gas, pyrolysis oil, or a combination of pyrolysis gas and pyrolysis oil, based on the total weight of the stream, or it can include these components in an amount in the range of from 1 to 95 weight percent, 5 to 90 weight percent, or 10 to 85 percent, based on the total weight of the stream.

In an embodiment or in combination with any embodiment mentioned herein, the cracker feed stream can include a hydrocarbon feed other than the pyrolysis gas and the pyrolysis oil stream in an amount of from 5 to 95 weight percent, 10 to 90 weight percent, or 15 to 85 weight percent, based on the total weight of the cracker feed. This hydrocarbon feed can comprise recycle content naphtha, recycle content propane, recycle content ethane, recycle content diesel, or a combination thereof. These additional hydrocarbon feed streams may be recovered from the downstream separator 18 within the cracking facility

In an embodiment or in combination with any embodiment mentioned herein, the cracker facility may comprise a single cracking furnace, or it can have at least 2, or at least 3, or at least 4, or at least 5, or at least 6, or at least 7, or at least 8 or more cracking furnaces operated in parallel. Any one or each furnace(s) may be gas cracker, or a liquid cracker, or a split furnace.

The cracker feed stream, along with the pyrolysis oil stream and/or pyrolysis gas, may pass through the cracking furnace, wherein the hydrocarbon components therein are thermally cracked to form lighter hydrocarbons, including olefins such as ethylene, propylene, and/or butadiene. The residence time of the cracker stream in the furnace can be in the range of from 0.15 to 2 seconds, 0.20 to 1.75 seconds, or 0.25 to 1.5 seconds.

The temperature of the cracked olefin-containing effluent withdrawn from the furnace outlet can be in the range of from 730 to 900° C., 750 to 875° C., or 750 to 850° C.

In an embodiment or in combination with any embodiment mentioned herein, the cracker furnace 16 may be at least partially heated by a combustion system comprising a plurality a burners that combust a combustion fuel and a combustion air. Furthermore, this combustion system may produce a flue gas stream that can be removed from the cracker furnace 16. The combustion fuel may comprise a conventional fossil fuel and/or a recycle content fuel, such as recycle content alkanes (e.g., r-methane) and/or recycle content hydrogen derived from the chemical recycling facility.

Upon exiting the cracker furnace 16 outlet, the olefin-containing effluent stream may be cooled rapidly (e.g., quenched) in the quench system (not shown) in order to prevent production of large amounts of undesirable by-products and to minimize fouling in downstream equipment. In an embodiment or in combination with any embodiment mentioned herein, the temperature of the olefin-containing effluent from the furnace can be reduced by 35 to 485° C., 35 to 375° C., or 90 to 550° C. to a temperature of 500 to 760° C. during the quench or cooling step.

The resulting cooled effluent stream can be then separated in a vapor-liquid separator, and the vapor can be compressed in a gas compressor having, for example, between 1 and 5 compression stages with optional inter-stage cooling and liquid removal. The pressure of the gas stream at the outlet of the first set of compression stages is in the range of from 7 to 20 bar gauge (barg), 8.5 to 18 barg, or 9.5 to 14 barg. The resulting compressed stream is then treated for removal of acid gases, including halogens, CO, CO₂, and H₂S by contact with an acid gas removal agent. Examples of acid gas removal agents can include, but are not limited to, caustic and various types of amines. In an embodiment or in combination with any embodiment mentioned herein, a single contactor may be used, while, in other embodiments, a dual column absorber-stripper configuration may be employed.

The treated compressed olefin-containing stream may then be further compressed in another compressor, optionally with inter-stage cooling and liquid separation. The resulting compressed stream, which has a pressure in the range of 20 to 50 barg, 25 to 45 barg, or 30 to 40 barg. Any suitable moisture removal method can be used including, for example, molecular sieves or other similar process. The resulting stream may then be passed to the separator 18 shown in FIG. 1 (e.g., a fractionation section), wherein the olefins and other components may be separated into various high-purity product or intermediate streams. In some embodiments, all or a portion of the pyrolysis gas may be introduced prior to and/or after one or more stages of the second compressor. Similarly, the pressure of the pyrolysis gas is within 20, within 50, within 100, or within 150 psi of the pressure of the stream with which it is being combined.

As used herein, the term “fractionation” refers to the general process of separating two or more materials having different boiling points. Examples of equipment and processes that utilize fractionation include, but are not limited to, distillation, rectification, stripping, and vapor-liquid separation (single stage).

In an embodiment or in combination with any embodiment mentioned herein, when introduced into the cracker facility, the pyrolysis gas stream may be introduced into the inlet of the cracker furnace 16, or all or a portion of the pyrolysis gas stream may be introduced downstream of the furnace outlet, at a location upstream of or within the separation zone of the cracker facility. When introduced into or upstream of the separation zone, the pyrolysis gas can be introduced upstream of the last stage of compression in the compressor, or prior to the inlet of at least one fractionation column in a fractionation section of the separator 18.

In an embodiment or in combination with any embodiment mentioned herein, the fractionation section of the separator 18 may include one or more of a demethanizer, a deethanizer, a depropanizer, an ethylene splitter, a propylene splitter, a debutanizer, and combinations thereof. As used herein, the term “demethanizer,” refers to a column whose light key component is methane. Similarly, “deethanizer,” and “depropanizer,” refer to columns with ethane and propane as the light key component, respectively.

Any suitable arrangement of columns may be used so that the fractionation section provides at least one olefin product stream and at least one paraffin stream. In an embodiment or in combination with any embodiment mentioned herein, the fractionation section can provide at least two olefin streams, such as ethylene and propylene, and at least two paraffin streams, such as ethane and propane, as well as additional streams including, for example, methane and lighter components and butane and heavier components.

In an embodiment or in combination with any embodiment mentioned herein, the recycle content olefin-containing stream withdrawn from the separator 18 in the cracking facility (as shown in FIG. 1) can comprise at least 10, at least 15, at least 20, at least 25, at least 30, at least 35, at least 40, at least 45, at least 50, at least 55, at least 60, at least 65, at least 70, at least 75, at least 80, at least 85, or at least 90 weight percent of recycle content C2 to C4 olefins, based on the total weight of the olefin-containing effluent stream. The recycle content olefin-containing stream may comprise predominantly recycle content ethylene, predominantly recycle content propylene, or predominantly recycle content ethylene and recycle content propylene, based on the total weight of the olefin-containing effluent stream.

In an embodiment or in combination with any embodiment mentioned herein, the olefin stream withdrawn from the separator 18 can comprise at least 50, at least 55, at least 60, at least 65, at least 70, at least 75, at least 80, at least 85, at least 90, or at least 95 weight percent and/or not more than 100, not more than 99, not more than 97, not more than 95, not more than 90, not more than 85, or not more than 80 weight percent of olefins, based on the total weight of the olefin stream. The olefins can be predominantly ethylene or predominantly propylene. The olefin stream can comprise at least 50, at least 55, at least 60, at least 65, at least 70, at least 75, at least 80, at least 85, at least 90, or at least 95 weight percent and/or not more than 99, not more than 97, not more than 95, not more than 90, not more than 85, not more than 80, not more than 75, not more than 70, or not more than 65 weight percent of ethylene, based on the total weight of olefins in the olefin stream. The olefin stream may comprise at least 20, at least 25, at least 30, at least 35, at least 40, at least 45, at least 50, at least 55, or at least 60 weight percent and/or not more than 80, not more than 75, not more than 70, not more than 65, not more than 60, not more than 55, not more than 50, or not more than 45 weight percent of ethylene, based on the total weight of the olefin stream, or it can be present in an amount in the range of from 20 to 80 weight percent, 25 to 75 weight percent, or 30 to 70 weight percent, based on the total weight of the olefin stream.

Alternatively, or in addition, the olefin stream can comprise at least 50, at least 55, at least 60, at least 65, at least 70, at least 75, at least 80, at least 85, at least 90, or at least 95 weight percent and/or not more than 99, not more than 97, not more than 95, not more than 90, not more than 85, not more than 80, not more than 75, not more than 70, or not more than 65 weight percent of propylene, based on the total weight of olefins in the olefin stream. In an embodiment or in combination with any embodiment mentioned herein, the olefin stream may comprise at least 20, at least 25, at least 30, at least 35, at least 40, at least 45, at least 50, at least 55, or at least 60 weight percent and/or not more than 80, not more than 75, not more than 70, not more than 65, not more than 60, not more than 55, not more than 50, or not more than 45 weight percent of propylene, based on the total weight of the olefin stream, or it can be present in an amount in the range of from 20 to 80 weight percent, 25 to 75 weight percent, or 30 to 70 weight percent, based on the total weight of the olefin stream.

As the compressed stream passes through the separator 18, it may pass through a demethanizer column, wherein the methane and lighter (CO, CO₂, H₂) components are separated from the ethane and heavier components. The demethanizer can be operated at a temperature of at least −145, or at least −142, or at least −140, or at least −135, in each case ° C. and/or not more than −120, not more than −125, not more than −130, not more than-135° C. The overhead stream from the demethanizer column may comprise at least 50, or at least 55, or at least 60, or at least 65, or at least 70, or at least 75, or at least 80, or at least 85, or at least 90, or at least 95 or at least 99 weight percent of methane, based on the total weight of the stream. This overhead stream may be removed from the cracking facility and be referred to as the recycle content methane (r-methane) stream.

Meanwhile, the bottoms stream from the demethanizer column may include at least 50, or at least 55, or at least 60, or at least 65, or at least 70, or at least 75, or at least 80, or at least 85, or at least 90, or at least 95 or at least 99, in each case percent of the total amount of ethane and heavier components.

In an embodiment or in combination with any embodiment mentioned herein, all or a portion of the stream introduced into the separator 18 can be introduced into a deethanizer column, wherein the C2 and lighter components are separated from the C3 and heavier components by fractional distillation. The deethanizer can be operated with an overhead temperature of at least −35, or at least −30, or at least −25, or at least −20, in each case ° C. and/or not more than −5, not more than −10, not more than −15, not more than −20° C., and an overhead pressure of at least 3, or at least 5, or at least 7, or at least 8, or at least 10, in each case barg and/or not more than 20, or not more than 18, or not more than 17, or not more than 15, or not more than 14, or not more than 13, in each case barg. The deethanizer column recovers at least 60, or at least 65, or at least 70, or at least 75, or at least 80, or at least 85, or at least 90, or at least 95, or at least 97, or at least 99, in each case percent of the total amount of C2 and lighter components introduced into the column in the overhead stream. The overhead stream removed from the deethanizer column comprises at least 50, or at least 55, or at least 60, or at least 65, or at least 70, or at least 75, or at least 80, or at least 85, or at least 90, or at least 95, in each case weight percent of ethane and ethylene, based on the total weight of the overhead stream.

In an embodiment or in combination with any embodiment mentioned herein, the C2 and lighter overhead stream from a deethanizer can be further separated in an ethane-ethylene fractionator column (ethylene fractionator or ethylene splitter). In the ethane-ethylene fractionator column, an ethylene and lighter component stream can be withdrawn from the overhead of the column or as a side stream from the top half of the column, while the ethane and any residual heavier components are removed in the bottoms stream. The ethylene fractionator may be operated at an overhead temperature of at least −45, or at least −40, or at least −35, or at least −30, or at least −25, or at least −20, in each case ° C. and/or not more than −15, or not more than −20, or not more than −25, in each case ° C., and an overhead pressure of at least 10, or at least 12, or at least 15, in each case barg and/or not more than 25, not more than 22, not more than 20 barg. The overhead stream, which may be enriched in ethylene, can include at least 70, or at least 75, or at least 80, or at least 85, or at least 90, or at least 95, or at least 97, or at least 98, or at least 99, in each case weight percent ethylene, based on the total weight of the stream and may be sent to downstream processing unit for further processing, storage, or sale. This removed ethylene may comprise recycle content ethylene (i.e., r-ethylene).

In an embodiment or in combination with any embodiment mentioned herein, at least a portion of the r-ethylene may be derived directly or indirectly from the cracking of r-pyoil and/or r-pyrolysis gas.

In some embodiments, at least a portion of the compressed stream may be separated in a depropanizer, wherein C3 and lighter components are removed as an overhead vapor stream, while C4 and heavier components exit the column in the liquid bottoms. The depropanizer can be operated with an overhead temperature of at least 20, or at least 35, or at least 40, in each case ° C. and/or not more than 70, 65, 60, 55° C., and an overhead pressure of at least 10, or at least 12, or at least 15, in each case barg and/or not more than 20, or not more than 17, or not more than 15, in each case barg. The depropanizer column recovers at least 60, or at least 65, or at least 70, or at least 75, or at least 80, or at least 85, or at least 90, or at least 95, or at least 97, or at least 99, in each case percent of the total amount of C3 and lighter components introduced into the column in the overhead stream. In an embodiment or in combination with any embodiment mentioned herein, the overhead stream removed from the depropanizer column comprises at least or at least 50, or at least 55, or at least 60, or at least 65, or at least 70, or at least 75, or at least 80, or at least 85, or at least 90, or at least 95, or at least 98, in each case weight percent of propane and propylene, based on the total weight of the overhead stream.

In an embodiment or in combination with any embodiment mentioned herein, the overhead stream from the depropanizer may be introduced into a propane-propylene fractionator (propylene fractionator or propylene splitter), wherein the propylene and any lighter components are removed in the overhead stream and the propane and any heavier components exit the column in the bottoms stream. The propylene fractionator may be operated at an overhead temperature of at least 20, or at least 25, or at least 30, or at least 35, in each case ° C. and/or not more than 55, not more than 50, not more than 45, not more than 40° C., and an overhead pressure of at least 12, or at least 15, or at least 17, or at least 20, in each case barg and/or not more than 20, or not more than 17, or not more than 15, or not more than 12, in each case barg. The overhead stream, which is enriched in propylene, can include at least 70, or at least 75, or at least 80, or at least 85, or at least 90, or at least 95, or at least 97, or at least 98, or at least 99, in each case weight percent propylene, based on the total weight of the stream and may be sent to downstream processing unit for further processing, storage, or sale.

In an embodiment or in combination with any embodiment mentioned herein, at least a portion of one or more of the above streams may be introduced into one or more of the facilities shown in FIG. 1, while, in other embodiments, all or a portion of the streams withdrawn from the separation zone of the cracking facility may be routed to further separation and/or storage, transportation, sale, and/or use.

### Recycle Content Ethylene Oxide Production and Recycle Content CO₂ Recovery

Turning back to FIG. 1, at least a portion of the r-ethylene stream from the cracking facility may be introduced into an ethylene oxide facility. While in the ethylene oxide facility, at least a portion of the r-ethylene may be oxidized in the presence of an oxygen stream to thereby yield a recycle content ethylene oxide (r-ethylene oxide) stream and a recycle content CO₂ (r-CO₂) stream.

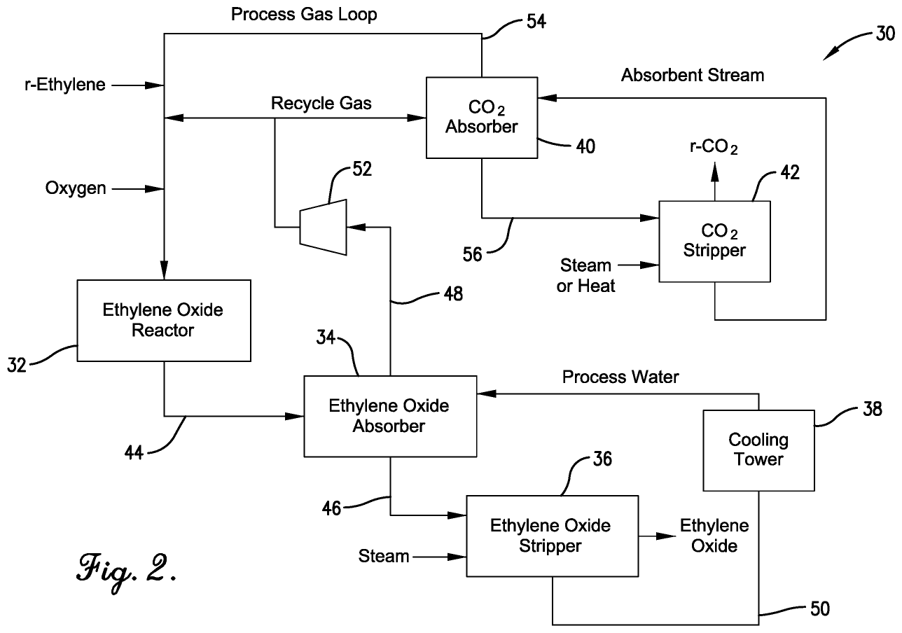

An exemplary ethylene oxide facility 30 is depicted in FIG. 2. As shown in FIG. 2, the ethylene oxide facility 30 may comprise at least one ethylene oxide reactor 32, at least one ethylene oxide absorber 34, at least one ethylene oxide stripper 36, at least one cooling tower 38, at least one CO₂ absorber 40, and at least one CO₂ stripper 42.

Ethylene oxide are important products in organic synthesis. Most ethylene oxide is used as an intermediate in the production of a large variety of other chemicals, such as alkanolamines, polyether polyols, and most notably ethylene glycol, which is used for the manufacture of polyesters, such as polyethylene terephthalate and copolyesters containing CHDM, neopentyl glycols, propylene glycols, or TMCD. As discussed below in greater detail, the process for producing an r-ethylene oxide composition can be generally carried out in a reaction vessel by charging one or more feedstock streams containing ethylene and oxygen into the reactor and reacting them in a direct oxidation method in the presence of a heterogenous catalyst.

As shown in FIG. 2, at least a portion of the r-ethylene can be subjected to a gas phase oxidation reaction step in the ethylene oxide reactor 32 using a supply of molecular oxygen and in the presence of a suitable catalyst, such as a silver catalyst, to thereby form an ethylene oxide vapor composition 44. Subsequently, the ethylene oxide vapor composition 44 can be transferred to an ethylene oxide absorber column, where the ethylene oxide vapor composition 44 is contacted with an absorption liquid (e.g., water) to produce a liquid (or aqueous) ethylene oxide composition. The resulting liquid ethylene oxide composition 46 may then be purified in an ethylene oxide stripper column 36 to obtain an enriched-liquid ethylene oxide composition enriched in ethylene oxide relative to the concentration of ethylene oxide discharged from the ethylene oxide absorber column.

During the oxidation reaction in the ethylene oxide reactor 32, at least a portion of the r-ethylene may be fed to the reactor, along with a molecular oxygen stream, which may comprise some additional gases, such as chlorinated compounds, nitrogen, helium, argon, carbon dioxide, steam, and/or C1-C3 alkanes. Inhibitors, such as chlorinated compounds (e.g., vinyl chloride, methyl chloride, t-butyl, monochloroethane, dichloromethane, and/or dichloroethylene) can also be added to the ethylene oxide reactor 32 in suitable amounts (e.g., 0.01-1000 ppm by volume) to act as reaction moderators and mitigate over-oxidation of ethylene oxide to CO₂ and H₂O.

In one embodiment or in combination with any of the mentioned embodiments, the ethylene feed into the ethylene oxide reactor 32 comprises at least 50, at least 75, at least 90, at least 95, at least 97, at least 98, or at least 99 weight percent of r-ethylene, based on the total weight of the ethylene feed stream.

Suitable reaction catalysts for the ethylene oxide reactor 32 may include silver metal or silver oxide deposited onto a solid carrier to make a heterogeneous catalyst. Suitable co-metal promoters or accelerators may include sodium, potassium, rubidium, rhenium, tungsten, molybdenum, chromium, cesium, and/or nitrate- or nitrite-forming compounds. Suitable supports may include alumina, aluminosilicates, magnesia, zirconia, silica, pumice, silicon carbide, and the like.

Suitable reaction temperatures for the ethylene oxide reactor 32 may range from 200 to 300° C. or 220 to 280° C., and care is taken to not over oxidize ethylene to CO₂ and thereby lower the yield of ethylene oxide. The reactor pressure can be from 150 to 440 psi and the reaction can be conducted at a residence time from 5 to 30 seconds or 5 to 15 seconds at gas hourly space velocities ranging from 100 to 20,000 hr⁻¹, 1,000 to 10,000 hr⁻¹, 2,000 to 8,000 hr⁻¹, or 3,000 to 7,000 hr⁻¹.

The oxygen supplied to the ethylene oxide reactor 32 can be air, but to increase the yield of ethylene oxide, it may be desirable to use a gaseous composition having a concentration of oxygen that is higher than atmospheric content, such as at least 50 mole percent purity, at least 80 mole percent purity, at least 90 mole percent purity, or at least 95 mole percent purity. The ethylene oxide reactor 32 can be a tubular reactor containing a plurality of tubes in a single or a plurality of bundles. For example, the reaction vessel can contain at least 20 tubes, at least 50 tubes, at least 100 tubes, at least 500 tubes, or at least 1,000 tubes. Each of the tubes can be packed with catalyst.

As shown in FIG. 2, at least a portion of the uncondensed gases discharged from the ethylene oxide absorber 34 may also contain some unreacted ethylene and/or oxygen and, therefore, can be reprocessed through the ethylene oxide reactor 32. In one embodiment or in any of the mentioned embodiments, unreacted ethylene can be recycled back to the ethylene oxide reactor 32, optionally taken from the overhead of the ethylene oxide absorber column.

The ethylene oxide stripper 36 may involve a separation step between light and heavy fractions and be facilitated by the introduction of a steam stream into the stripper. The liquid or aqueous ethylene oxide composition 46 discharged from the ethylene oxide absorber column 34 can be charged to an ethylene oxide stripper column 36 to obtain an ethylene oxide gaseous overhead 48. While in the ethylene oxide stripper 36, the ethylene oxide can be stripped of its low boilers and the remaining ethylene oxide can be distilled to separate water from ethylene oxide. The residual liquid stream 50 from the ethylene oxide stripper 36, which comprises a substantial amount of process water, may be sent to a cooling tower 38, where it is further cooled, prior to reintroduction into the ethylene oxide absorber column 34.

As shown in FIG. 2, unreacted ethylene can be discharged from the ethylene oxide reactor 32 and be sent to the ethylene oxide absorber column 34. Subsequently, any remaining unreacted ethylene 48 may be discharged from the overhead of the ethylene oxide absorber column 34, along with CO₂, water, and inert gases. Optionally, at least a portion of the ethylene oxide absorber overhead stream 48 can be recycled back to the ethylene oxide reactor 32 for the oxidation step.

Additionally, or in the alternative, at least a portion of the overhead stream 48 from the ethylene oxide absorber column 34 can be purged and fed to a CO₂ absorber column 40 to contact the CO₂-containing gas with an absorber solvent in order to strip and recover a recycle content CO₂ stream.

As shown in FIG. 2, at least a portion of the CO₂ contained in the overhead gas stream from the ethylene oxide absorber column 34 can be recovered. At least a portion of the ethylene oxide absorber column 34 overhead stream 48 can be compressed in a compressor 52 and fed to a CO₂ absorber column 40 in which the overhead stream is contacted, optionally in a countercurrent flow, with an absorber solvent (e.g., a hot aqueous alkali solution such as potassium carbonate) to thereby form a rich absorber solvent enriched in CO₂ as an underflow. Generally, the absorber solvent can comprise an organic solvent. In an embodiment or in combination with any embodiment mentioned herein, the absorber solvent can comprise an absorbing component selected from the group consisting of amines, methanol, sodium hydroxide, sodium carbonate/bicarbonate, potassium hydroxide, potassium carbonate/bicarbonate, SELEXOL®, glycol ether, and combinations thereof. The absorbing component may comprise an amine selected from the group consisting of diethanolamine (DEA), monoethanolamine (MEA), methyldiethanolamine (MDEA), diisopropanolamine (DIPA), diglycolamine (DGA), piperazine, modifications, derivatives, and combinations thereof.

Furthermore, as shown in FIG. 2, an overhead stream 54 comprising unreacted ethylene may be removed from CO₂ absorber column 40 and recycled back to the ethylene oxide reactor 32 as the process gas loop.

The underflow 56 can then be charged to a CO₂ stripper column 42 where at least a portion of the CO₂ is liberated from the absorber solvent, typically stepwise by flashing the underflow with water/steam. The flashing can be generated by operating the CO₂ stripper column 42 under a pressure that is less than the pressure in the CO₂ absorber column 40. Suitable pressure in the CO₂ stripper column 42 can be from 0.01 to 0.5 MPa gauge. The CO₂ stripper column 42 can be operated at a temperature from 80 to 120° C.

The recovered r-CO₂ gas may be removed as the recycled r-CO₂ stream. After CO₂ removal, the resulting lean absorber solvent may be recycled back to the CO₂ absorber column 40. Thus, a regenerative absorber system can be used between the CO₂ absorber column 40 and the CO₂ stripper column 42, where the rich absorber solvent stream is sent from the CO₂ absorber column 40 to the CO₂ stripper column 42 and the lean absorber solvent stream is returned from the CO₂ stripper column 42 to the CO₂ absorber column 40.

In an embodiment or in combination with any embodiment mentioned herein, the recovered r-CO₂ stream comprises at least 5, at least 10, at least 15, at least 20, at least 25, at least 30, at least 35, at least 40, at least 45, at least 50, at least 75, at least 80, at least 85, at least 90, at least 95, at least 96, at least 97, at least 98, or at least 99 mole percent of CO₂, based on the total molar content of the recovered CO₂ stream. In such embodiments, at least a portion or all of the recovered CO₂ in the recovered CO₂ stream may be considered recycle content CO₂ since it may be directly and/or indirectly derived from waste plastic. Thus, the recovered CO₂ stream may be referred to as a recovered r-CO₂ stream that comprises at least 5, at least 10, at least 15, at least 20, at least 25, at least 30, at least 35, at least 40, at least 45, at least 50, at least 75, at least 80, at least 85, at least 90, at least 95, at least 96, at least 97, at least 98, or at least 99 mole percent of r-CO₂, based on the total molar content of the recovered CO₂ stream.

In an embodiment or in combination with any embodiment mentioned herein, the recovered CO₂ stream comprises at least 0.1, at least 0.5, at least 1, at least 2, or at least 2.5 and/or not more than 10, not more than 5, not more than 4, or not more than 3 mole percent of water, based on the total molar content of the recovered CO₂ stream.

In an embodiment or in combination with any embodiment mentioned herein, the recovered CO₂ stream comprises not more than 1,000 ppm, not more than 500 ppm, or not more than 200 ppm of an absorber solvent, based on the total molar content of the recovered CO₂ stream.

In an embodiment or in combination with any embodiment mentioned herein, the recovered CO₂ stream has a temperature of at least 25, at least 30, at least 40, at least 45, or at least 50 and/or not more than not more than 100, not more than 75, not more than 70, not more than 65, or not more than 60° C. upon exiting the CO₂ stripper column 42.

As discussed below, the recovered r-CO₂ stream may be introduced into a molecular reforming facility, as shown in FIG. 1, in order to produce a recycle content syngas (r-syngas).

### Molecular Reforming

Turning back to FIG. 1, at least a portion of the recovered r-CO₂ stream may be introduced into the molecular reforming facility comprising a gasifier 22. Exemplary molecular reforming facilities can include a partial oxidation (POX) gasification facility or a steam reforming facility. In an embodiment or in combination with any embodiment mentioned herein, the molecular reforming facility may comprise a POX gasifier.

The reactions occurring within molecular reforming facility include conversion of the gasifier feedstock into syngas, and specific examples include, but are not limited to partial oxidation, water gas shift, water gas-primary reactions, Boudouard, oxidation, methanation, hydrogen reforming, steam reforming, and carbon dioxide reforming.

In an embodiment or in combination with any embodiment mentioned herein, the feed to the molecular reforming facility can comprise a recycle content feed component (e.g., r-pyrolysis effluent, r-pyoil, and/or r-pygas) and/or a non-recycle content feed component (e.g., coal, a liquid hydrocarbon, and/or a gaseous hydrocarbon), in addition to the recovered r-CO₂ stream.

In an embodiment or in combination with any embodiment mentioned herein, the feed to the molecular reforming facility can comprise a hydrocarbon (e.g., a liquid hydrocarbon, a solid hydrocarbon, and/or a gaseous hydrocarbon), water, steam, pyrolysis gas, pyrolysis oil, pyrolysis residue, and/or natural gas, in addition to the recovered r-CO₂ stream.

In an embodiment or in combination with any embodiment mentioned herein, the molecular reforming is partial oxidation gasification that is fed with coal and waste plastic. In yet other embodiments, the molecular reforming is plasma gasification of a predominately waste plastic feed. In yet even other embodiments, the molecular reforming is partial oxidation gasification fed with a non-recycle content liquid or gaseous hydrocarbon and a recycle content pyrolysis oil produced from the pyrolysis of waste plastic. In some embodiments, the molecular reforming can include catalytic reforming, while in other embodiments, the carbon reforming can include steam reforming.

In an embodiment or in combination with any embodiment mentioned herein, the molecular reforming facility comprises a POX gasification facility. The feed to POX gasification can include solids, liquids, and/or gases. When multiple types of feed streams are present, each may be introduced separately, or all or a portion of the streams may be combined so that the combined stream may be introduced into the POX gasification facility. The combining, when present, may take place in a continuous or batch manner. The feed stream can be in the form of a gas, a liquid or liquified plastic, solids (usually comminuted), or a slurry.

In the POX gasification facility, at least a portion of the recovered r-CO₂ stream, along with other gasifier feed streams, may be converted to syngas in the presence of a sub-stoichiometric amount of oxygen. The POX gasification facility includes at least one POX gasification reactor.

In an embodiment or in combination with any embodiment mentioned herein, the feed stream to the POX gasification facility may comprise at least 50, at least 55, at least 60, at least 65, at least 70, at least 75, at least 80, at least 85, at least 90, at least 95, at least 99, or at least 99.5 weight percent of a waste plastic, a hydrocarbon (solid, liquid, and/or gaseous), a pyrolysis product (e.g., the r-pyoil and/or r-pygas), natural gas, and/or the recovered r-CO₂ stream, based on the total weight of the gasifier feed stream. Furthermore, in one or more embodiments, the liquefied waste plastic may be introduced into the POX gasification facility at a rate of at least 1,000, at least 5,000, at least 10,000, at least 20,000, at least 40,000, at least 80,000, or at least 120,000 lbs/hour.

Exemplary fuels that may be introduced into the POX gasifier and/or initially combined with the recovered r-CO₂ stream may include, for example, a solid (e.g., coal, petrocoke, waste plastics, etc.), liquid (e.g., liquid hydrocarbons, liquefied plastics, etc.), and/or a gas (e.g., natural gas, hydrocarbons, etc.). As used herein, a “gasification feedstock” or “gasifier feed” refers to all components fed into the gasifier except oxygen.

In addition to the recovered r-CO₂ stream, in an embodiment or in combination with any embodiment mentioned herein, the gasification feedstock stream may also comprise at least 1, at least 5, at least 10, at least 15, at least 20, at least 25, at least 30, at least 35, at least 40, at least 45, or at least 50 weight percent of one or more solid fuels, based on the total weight of the gasification feedstock stream. Additionally, or in the alternative, the gasification feedstock stream may also comprise not more than 99, not more than 90, not more than 80, not more than 70, not more than 60, not more than 50, not more than 40, not more than 30, not more than 20, not more than 10, not more than 5, not more than 4, not more than 3, not more than 2, or not more than 1 weight percent of one or more solid fuels, based on the total weight of the gasification feedstock stream. Exemplary solid fuels can include coal.

In certain embodiments, the gasification feedstock stream does not contain any solid fuels (i.e., feeds that are solid at 25° C. and 1 atm). Thus, in such embodiments, the feed to the gasifier 22 and the gasification reactions will not contain any solid feeds.

In addition to the recovered r-CO₂ stream, in an embodiment or in combination with any embodiment mentioned herein, the gasification feedstock stream may also comprise at least 1, at least 5, at least 10, at least 15, at least 20, at least 25, at least 30, at least 35, at least 40, at least 45, or at least 50 weight percent of one or more liquid fuels, based on the total weight of the gasification feedstock stream. Additionally, or in the alternative, the gasification feedstock stream may also comprise not more than 99, not more than 90, not more than 80, not more than 70, not more than 60, not more than 50, not more than 40, not more than 30, not more than 20, not more than 10, not more than 5, not more than 4, not more than 3, not more than 2, or not more than 1 weight percent of one or more liquid fuels, based on the total weight of the gasification feedstock stream. Exemplary liquid fuels can include liquid hydrocarbons, liquefied plastics, and/or at least partially condensed r-pyoil.

In addition to the recovered r-CO₂ stream, in an embodiment or in combination with any embodiment mentioned herein, the gasification feedstock stream may also comprise at least 1, at least 5, at least 10, at least 15, at least 20, at least 25, at least 30, at least 35, at least 40, at least 45, or at least 50 weight percent of one or more gas fuels, based on the total weight of the gasification feedstock stream. Additionally, or in the alternative, the gasification feedstock stream may also comprise not more than 99, not more than 90, not more than 80, not more than 70, not more than 60, not more than 50, not more than 40, not more than 30, not more than 20, not more than 10, not more than 5, not more than 4, not more than 3, not more than 2, or not more than 1 weight percent of one or more gas fuels, based on the total weight of the gasification feedstock stream. Exemplary gas fuels can include r-pygas, natural gas, and/or gaseous hydrocarbons.

In an embodiment or in combination with any embodiment mentioned herein, a gas stream enriched in hydrogen (H₂) (e.g., at least 1, at least 2, at least 5, at least 10, at least 20, at least 30, at least 40, at least 50, at least 60, at least 70, at least 80, or at least 90 mole percent is charged into the gasifier 22. Hydrogen may be added to affect the partial oxidation reactions so as to control the resulting syngas composition.

In an embodiment or in combination with any embodiment mentioned herein, the gasification feedstock stream may comprise an oxygen/carbon molar ratio in the range of 0.5 to 1.5, 0.6 to 1.3, or 0.7 to 1.1.

In an embodiment or in combination with any embodiment mentioned herein, the recovered r-CO₂ stream may serve as carrier gas to propel a feedstock to a gasification zone. Due to the pressure within the gasification zone, the recovered r-CO₂ stream may be compressed to provide the motive force for introduction into the gasification zone.

Furthermore, the POX gasification unit may comprise a gas-fed, a liquid-fed, or a solid-fed reactor (or gasifier).

The POX gasification unit may comprise a gas-fed reactor (or gasifier). In an embodiment or in combination with any embodiment mentioned herein, the POX gasification facility may perform gas-fed POX gasification. As used herein, “gas-fed POX gasification” refers to a POX gasification process where the feed to the process comprises predominately (by weight) components that are gaseous at 25° C. and 1 atm.

Gas-fed POX gasification processes can be co-fed with lesser amounts of other components having a different phase at 25° C. and 1 atm. Thus, gas-fed POX gasifiers can be co-fed with liquids and/or solids, but only in amounts that are less (by weight) than the amount of gases fed to the gas-phase POX gasifier.

In an embodiment or in combination with any embodiment mentioned herein, the POX gasification facility may perform liquid-fed POX gasification. As used herein, “liquid-fed POX gasification” refers to a POX gasification process where the feed to the process comprises predominately (by weight) components that are liquid at 25° C. and 1 atm.

Additionally, or alternatively, POX gasification unit may conduct solid-fed POX gasification. As used herein, “solid-fed POX gasification” refers to a POX gasification process where the feed to the process comprises predominately (by weight) components that are solid at 25° C. and 1 atm.

Gas-fed, liquid-fed, and solid-fed POX gasification processes can be co-fed with lesser amounts of other components having a different phase at 25° C. and 1 atm. Thus, gas-fed POX gasifiers can be co-fed with liquids and/or solids, but only in amounts that are less (by weight) than the amount of gasses fed to the gas-phase POX gasifier; liquid-fed POX gasifiers can be co-fed with gasses and/or solids, but only in amounts (by weight) less than the amount of liquids fed to the liquid-fed POX gasifier; and solid-fed POX gasifiers can be co-fed with gasses and/or liquids, but only in amounts (by weight) less than the amount of solids fed to the solid-fed POX gasifier.

In an embodiment or in combination with any embodiment mentioned herein, the total feed to a gas-fed POX gasifier can comprise at least 60, at least 70, at least 80, at least 90, or at least 95 weight percent of components that are gaseous at 25° C. and 1 atm; the total feed to a liquid-fed POX gasifier can comprise at least 60, at least 70, at least 80, at least 90, or at least 95 weight percent of components that are liquid at 25° C. and 1 atm; and the total feed to a solid-fed POX gasifier can comprise at least 60, at least 70, at least 80, at least 90, or at least 95 weight percent of components that are solids at 25° C. and 1 atm.

In an embodiment or in combination with any embodiment mentioned herein, the type of gasification technology employed may be a partial oxidation entrained flow gasifier that generates syngas. This technology is distinct from fixed bed (alternatively called moving bed) gasifiers and from fluidized bed gasifiers. An exemplary gasifier that may be used is depicted in U.S. Pat. No. 3,544,291, the entire disclosure of which is incorporated herein by reference to the extent not inconsistent with the present disclosure. However, in an embodiment or in combination with any embodiment mentioned herein, other types of gasification reactors may also be used within the scope of the present technology.

In an embodiment or in combination with any embodiment mentioned herein, the gasifier/gasification reactor can be non-catalytic, meaning that the gasifier/gasification reactor does not contain a catalyst bed and the gasification process is non-catalytic, meaning that a catalyst is not introduced into the gasification zone as a discrete unbound catalyst. Furthermore, in an embodiment or in combination with any embodiment mentioned herein, the gasification process may not be a slagging gasification process; that is, operated under slagging conditions (well above the fusion temperature of ash) such that a molten slag is formed in the gasification zone and runs along and down the refractory walls.

In an embodiment or in combination with any embodiment mentioned herein, the gasification zone, and optionally all reaction zones in the gasifier/gasification reactor, may be operated at a temperature of at least 1000° C., at least 1100° C., at least 1200° C., at least 1250° C., or at least 1300° C. and/or not more than 2500° C., not more than 2000° C., not more than 1800° C., or not more than 1600° C. The reaction temperature may be autogenous. Advantageously, the gasifier operating in steady state mode may be at an autogenous temperature and does not require application of external energy sources to heat the gasification zone.

In an embodiment or in combination with any embodiment mentioned herein, the gasification zone, and optionally all reaction zones in the gasifier/gasification reactor, may comprise a sidewall temperature of at least 1000° C., at least 1100° C., at least 1200° C., at least 1250° C., or at least 1300° C. and/or not more than 2500° C., not more than 2000° C., not more than 1800° C., not more than 1600° C., or not more than 1500° C.

Generally, the gasification feed stream may be introduced into a gasification reactor along with an oxidizing agent stream. The feedstock stream and the oxidizing agent stream may be sprayed through an injector assembly into a pressurized gasification zone having, for example, a pressure, typically at least 500, at least 600, at least 800, or at least 1,000 psig, (or at least 35, at least 40, at least 55, or at least 70 barg). In an embodiment or in combination with any embodiment mentioned herein, the injector assembly may comprise a single injector or a plurality of injectors.

In an embodiment or in combination with any embodiment mentioned herein, the injection assembly may be positioned at the top of gasifier or near the top of the gasifier. In certain embodiments, when a gaseous gasifier feed is utilized, at least a portion of the gaseous gasifier feed (e.g., natural gas) may be fed into the gasifier via a side draw injector and is not fed via an injector positioned at the top of the gasifier.

The oxidizing agent can include other oxidizing gases or liquids, in addition to or in place of air, oxygen-enriched air, and molecular oxygen. Examples of such oxidizing liquids suitable for use as oxidizing agents include water (which can be added as a liquid or as steam) and ammonia. Examples of such oxidizing gases suitable for use as oxidizing agents include carbon monoxide, carbon dioxide, and sulfur dioxide.

In an embodiment or in combination with any embodiment mentioned herein, an atomization enhancing fluid is fed to the gasification zone along with the feedstock and oxidizing agent. As used herein, the term “atomization enhancing fluid” refers to a liquid or gas operable to reduce viscosity to decrease dispersion energy, or increase energy available to assist dispersion. The atomization enhancing fluid may be mixed with the gasifier feedstock before the feedstock is fed into the gasification zone or separately added to the gasification zone, for example to an injection assembly coupled with the gasification reactor. In an embodiment or in combination with any embodiment mentioned herein, the atomization enhancing fluid is water and/or steam. However, in an embodiment or in combination with any embodiment mentioned herein, steam and/or water is not supplied to the gasification zone.

Mixing of the feedstock stream and the oxidizing agent may be accomplished entirely within the reaction zone by introducing the separate streams of feedstock and oxidizing agent so that they impinge upon each other within the reaction zone. In an embodiment or in combination with any embodiment mentioned herein, the oxidizing agent stream is introduced into the reaction zone of the gasifier as high velocity to both exceed the rate of flame propagation and to improve mixing with the feedstock stream. In an embodiment or in combination with any embodiment mentioned herein, the oxidant may be injected into the gasification zone in the range of 25 to 500, 50 to 400, or 100 to 400 feet per second. These values would be the velocity of the gaseous oxidizing agent stream at the injector-gasification zone interface, or the injector tip velocity. Mixing of the feedstock stream and the oxidizing agent may also be accomplished outside of the reaction zone. For example, in an embodiment or in combination with any embodiment mentioned herein, the feedstock, oxidizing agent, and/or atomization enhancing fluid can be combined in a conduit upstream of the gasification zone or in an injection assembly coupled with the gasification reactor.

In an embodiment or in combination with any embodiment mentioned herein, the gasifier may be operated at a pressure within the gasification zone (or combustion chamber) of at least 200 psig (1.38 MPa), at least 300 psig (2.06 MPa), at least 350 psig (2.41 MPa), at least 400 psig (2.76 MPa), at least 420 psig (2.89 MPa), at least 450 psig (3.10 MPa), at least 475 psig (3.27 MPa), at least 500 psig (3.44 MPa), at least 550 psig (3.79 MPa), at least 600 psig (4.13 MPa), at least 650 psig (4.48 MPa), at least 700 psig (4.82 MPa), at least 750 psig (5.17 MPa), at least 800 psig (5.51 MPa), at least 900 psig (6.2 MPa), at least 1000 psig (6.89 MPa), at least 1100 psig (7.58 MPa), or at least 1200 psig (8.2 MPa). Additionally or alternatively, the gasifier may be operated at a pressure within the gasification zone (or combustion chamber) of not more than 1300 psig (8.96 MPa), not more than 1250 psig (8.61 MPa), not more than 1200 psig (8.27 MPa), not more than 1150 psig (7.92 MPa), not more than 1100 psig (7.58 MPa), not more than 1050 psig (7.23 MPa), not more than 1000 psig (6.89 MPa), not more than 900 psig (6.2 MPa), not more than 800 psig (5.51 MPa), or not more than 750 psig (5.17 MPa).

In an embodiment or in combination with any embodiment mentioned herein, the gasifier is a non-slagging gasifier or operated under conditions not to form a slag.

In an embodiment or in combination with any embodiment mentioned herein, the gasifier may comprise a fixed bed gasifier. Alternatively, in an embodiment or in combination with any embodiment mentioned herein, the gasifier may not contain a gasifier bed, such as fixed bed gasifier.

In an embodiment or in combination with any embodiment mentioned herein, the gasifier may comprise a refractory-lined gasifier.

In an embodiment or in combination with any embodiment mentioned herein, the gasifier may comprise a downflow gasifier. In such embodiments, the gasifier feed can be introduced at or near the top of the gasifier and allowed to flow down the reactor for the gasification reaction.

In an embodiment or in combination with any embodiment mentioned herein, the gasifier may comprise a single stage gasifier. In such embodiments, the gasifier may comprise a single gasification stage.

In an embodiment or in combination with any embodiment mentioned herein, the gasifier does not comprise any pyrolysis zones or pyrolysis stages. More particularly, the gasifier may not contain any reaction zones or stages operating at the pyrolysis conditions described herein, including the pyrolysis temperatures and residence times. Thus, in such embodiments, all of the gasification zones/stages within the gasifier operate at conditions that are not within the conditions for pyrolysis.

Generally, the average residence time of gases in the gasifier reactor can be very short to increase throughput. Since the gasifier may be operated at high temperature and pressure, substantially complete conversion of the feedstock to gases can occur in a very short time frame. In an embodiment or in combination with any embodiment mentioned herein, the average residence time of the gases in the gasifier can be not more than 30, not more than 25, not more than 20, not more than 15, not more than 10, or not more than 7 seconds.

Generally, the recycle content syngas stream discharged from the gasification vessel includes such gases as hydrogen, carbon monoxide, and carbon dioxide and can include other gases such as methane, hydrogen sulfide, and nitrogen depending on the fuel source and reaction conditions.

In an embodiment or in combination with any embodiment mentioned herein, the recycle content syngas stream (r-syngas) (the stream discharged from the gasifier and before any further treatment by way of scrubbing, shift, or acid gas removal) can have the following composition in mole percent on a dry basis and based on the moles of all gases (elements or compounds in gaseous state at 25° C. and 1 atm) in the r-syngas stream:
a hydrogen content in the range of 32 to 50 percent, or at least 33, at least 34, or at least 35 and/or not more than 50, not more than 45, not more than 41, not more than 40, or not more than 39 percent, or it can be in the range of 33 to 50 percent, 34 to 45 percent, or 35 to 41 percent, on a dry volume basis;
a carbon monoxide content of at least 40, at least 41, at least 42, or at least 43 and/or not more than 55, not more than 54, not more than 53, or not more than 52 weight percent, based on the total weight of the stream, or in the range of from 40 to 55 weight percent, 41 to 54 weight percent, or 42 to 53 weight percent, based on the total weight of the stream on a dry basis;
a carbon dioxide content of at least 1%, at least 1.5%, at least 2%, at least 3%, at least 4%, at least 5%, at least 6%, or at least 7% by volume and/or not more than 25%, not more than 20%, not more than 15%, not more than 12%, not more than 11%, not more than 10%, not more than 9%, not more than 8%, or not more than 7% by volume on a dry basis;
a methane content of not more than 5000, not more than 2500, not more than 2000, or not more than 1000 ppm by volume methane on a dry basis;
a sulfur content of not more than 1000, not more than 100, not more than 10, or not more than 1 ppm by weight (ppmw);
a soot content of at least 1000, or at least 5000 ppm and/or not more than 50,000, not more than 20,000, or not more than 15,000 ppmw;
a halides content of not more than 1000, not more than 500, not more than 200, not more than 100, or not more than 50 ppmw;
a mercury content of not more than 0.01, not more than 0.005, or not more than 0.001 ppmw;
an arsine content of not more than 0.1 ppm, not more than 0.05, or not more than 0.01 ppmw;
a nitrogen content of not more than 10,000, not more than 3000, not more than 1000, or not more than 100 ppmw nitrogen;
an antimony content of at least 10 ppmw, at least 20 ppmw, at least 30 ppmw, at least 40 ppmw, or at least 50 ppmw, and/or not more than 200 ppmw, not more than 180 ppmw, not more than 160 ppmw, not more than 150 ppmw, or not more than 130 ppmw; and/or
a titanium content of at least 10 ppmw, at least 25 ppmw, at least 50 ppmw, at least 100 ppmw, at least 250 ppmw, at least 500 ppmw, or at least 1000 ppmw, and/or not more than 40,000 ppmw, not more than 30,000 ppmw, not more than 20,000 ppmw, not more than 15,000 ppmw, not more than 10,000 ppmw, not more than 7,500 ppmw, or not more than 5,000 ppmw.

In an embodiment or in combination with any embodiment mentioned herein, the r-syngas comprises a molar hydrogen/carbon monoxide ratio of at least 0.5, at least 0.6, at least 0.7, at least 0.8, or at least 0.9 and/or less than 2, less than 1.9, less than 1.8, less than 1.7, less than 1.6, less than 1.5, less than 1.4, less than 1.3, less than 1.2, less than 1.1, or less than 1.05. For example, the r-syngas comprises a molar hydrogen/carbon monoxide ratio of 0.5 to 2, 0.7 to 2, 0.5 to 1.7, 0.7 to 1.7, 0.7 to 1.5, 0.8 to 1.2, 0.85 to 1.1, or 0.9 to 1.05.

The gas components can be determined by Flame Ionization Detector Gas Chromatography (FID-GC) and Thermal Conductivity Detector Gas Chromatography (TCD-GC) or any other method recognized for analyzing the components of a gas stream.

The raw syngas stream discharged from the gasification vessel may be further processed in a sulfur/nitrogen oxide (“NOx”) removal zone to remove sulfur, nitrogen oxides, and sulfur-containing compounds from the raw syngas, so as to produce a purified syngas stream. Any conventional sulfur removal technique known in the art may be used.

Additionally, the raw syngas stream, before or after sulfur removal, may be cooled in a quench system, such as a water bath. This quenching step may cool the r-syngas stream and set the equilibrium of the water-gas-shift reaction.

The resulting r-syngas stream may be removed from the chemical recycling facility 10 for sale or use in other downstream facilities not depicted in FIG. 1. In certain embodiments, at least a portion of the r-syngas stream may be used in downstream facilities to produce various chemical derivatives and/or subjected to further processing and purification in order to obtain recycle content hydrogen and/or recycle content methane therefrom.

CO₂ Recovery from Flue Gas Streams

As noted above, the liquification vessel, the pyrolysis reactor 14, and/or the cracker furnace 16 may be at least partially heated by a combustion system comprising a plurality a burners that combust a combustion fuel and a combustion air. The combustion fuel may comprise a conventional fossil fuel and/or a recycle content fuel, such as recycle content alkanes (e.g., r-methane) and/or recycle content hydrogen derived from the chemical recycling facility.

Furthermore, these combustion systems may produce flue gas streams from which additional CO₂ may be recovered and further gasified in the gasifier so as to produce additional recycle content syngas. Moreover, in certain embodiments, these combustion systems may utilize at least a portion of the r-methane produced in the cracking facility as the combustion fuel used to the flue gas streams.

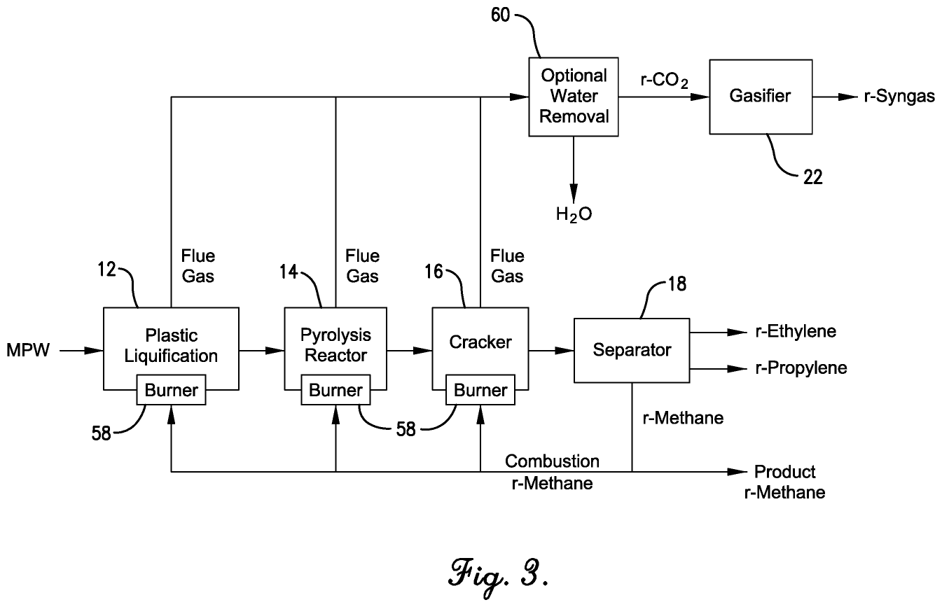

An exemplary facility utilizing combustion devices 58 for the liquification vessel, the pyrolysis reactor 14, and/or the cracker furnace 16 is depicted in FIG. 3. It should be noted that the process steps shown in FIG. 3 may operate in the same manner as described above in regard to FIGS. 1 and 2, unless otherwise noted. Thus, all of the reaction step and conditions described above in regard to FIGS. 1 and 2 are also applicable to the embodiment depicted in FIG. 3, unless otherwise noted.

As shown in FIG. 3, the liquification vessel in the plastic liquification system 12, the pyrolysis reactor 14, and the cracker furnace 16 each have a combustion device 58 comprising at least one burner. The flue gas streams may be recovered from each of these combustion devices and further treated in an optional water removal step 60 to remove any residual water present in the flue gas streams, thereby leaving an enriched r-CO₂ stream. Upon leaving the optional water removal step 60, the enriched r-CO₂ stream may be introduced and treated in the molecular reforming facility, as described above, in order to produce the r-syngas stream.

Furthermore, as shown in FIG. 3, at least a portion of the r-methane formed in the cracking facility may be recovered and either: (1) sold as a downstream product and/or (2) used as a combustion fuel for the burners in the combustion devices of the liquification vessel in the plastic liquification system 12, the pyrolysis reactor 14, and/or the cracker furnace 16. In an embodiment or in combination with any embodiment mentioned herein, at least a portion of the r-methane formed in the cracking facility is used as the combustion fuel for the burners in the combustion devices of the liquification vessel in the plastic liquification system 12, the pyrolysis reactor 14, and/or the cracker furnace 16. In such embodiments, the combustion fuel for the combustion devices may comprise at least 50, at least 60, at least 70, at least 80, least 90, at least 95, or at least 99 weight percent of r-methane, based on the total weight of the combustion fuel.

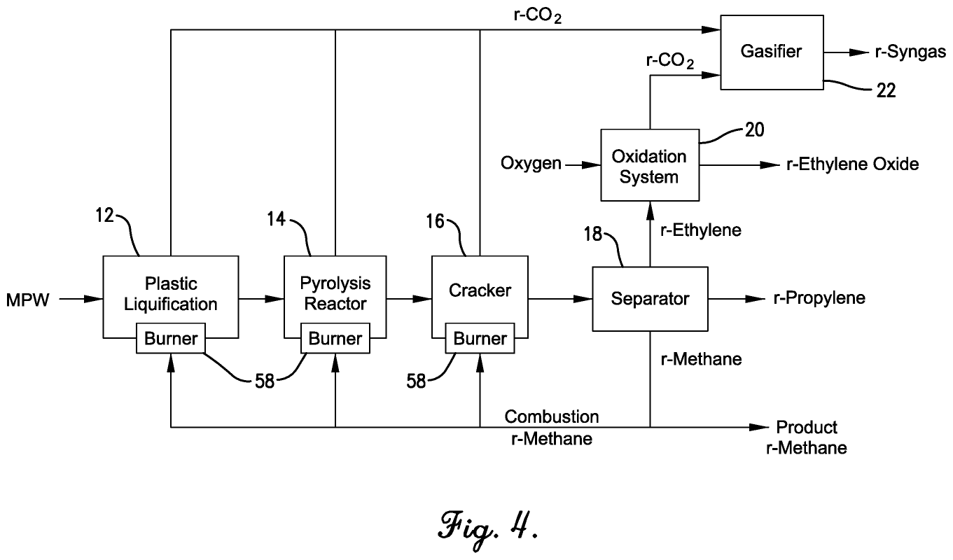

FIG. 4 depicts yet another exemplary embodiment of how CO₂ may be recovered from the combustion of at least a portion of the r-methane stream from the cracking facility. It should be noted that the process steps shown in FIG. 4 may operate in the same manner as described above in regard to FIGS. 1-3, unless otherwise noted. Thus, all of the reaction step and conditions described above in regard to FIGS. 1-3 are also applicable to the embodiment depicted in FIG. 4, unless otherwise noted.

The system configuration in FIG. 4 operates in the same manner as in FIG. 3, except the optional water removal step is omitted and the flue gas streams (containing the r-CO₂) can be introduced directly into the molecular reforming facility. In addition, FIG. 4 shows how the r-CO₂ may also be recovered from the ethylene oxide facility, as described above.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, “aqueous” refers to a fluid containing at least five percent of molecular water by weight.

As used herein, the term “bottom” refers to the physical location of a structure that is below the other noted structures within an enclosed structure. For example, a “bottom” stream is a stream taken from a vessel at a position that is lower elevation-wise to other streams taken from the vessel, such as an “overhead” stream.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the term “chemical recycling facility” refers to a facility for producing a recycle content product via chemical recycling of waste plastic.

As used herein, the term “co-located” refers to the characteristic of at least two objects being situated on a common physical site, and/or within one mile of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the term “depleted” refers to having a concentration (on a dry weight basis) of a specific component that is less than the concentration of that component in a reference material or stream.

As used herein, the term “directly derived” refers to having at least one physical component originating from waste plastic.

As used herein, the term “enriched” refers to having a concentration (on a dry weight basis) of a specific component that is greater than the concentration of that component in a reference material or stream.

As used herein, the term “ethylene oxide facility” refers to a facility that includes all equipment, lines, and controls necessary to produce ethylene oxide and generate a CO₂ stream.

As used herein, the terms “exhaustion” or “exhausting” refer to methods for disposing of the specified stream by removing the stream from the facility. Exemplary exhaustion methods can include venting.

As used herein, the term “fluid” may encompass a liquid, a gas, a supercritical fluid, or a combination thereof.

As used herein, the term “halide” refers to a composition comprising a halogen atom bearing a negative charge (i.e., a halide ion).

As used herein, the term “halogen” or “halogens” refers to organic or inorganic compounds, ionic, or elemental species comprising at least one halogen atom.

As used herein, the terms “having,” “has,” and “have” have the same open-ended meaning as “comprising,” “comprises,” and “comprise” provided above.

As used herein, a “heat transfer medium loop” or “HTM loop” refers to a system comprising one or more heat exchangers through which a common HTM is circulated to a common HTM supply or a part of a larger system for the purpose of transferring heat and/or energy into and/or out of the chemical recycling process.

As used herein, the terms “including,” “include,” and “included” have the same open-ended meaning as “comprising,” “comprises,” and “comprise” provided above.

As used herein, the term “indirectly derived” refers to having an assigned recycle content i) that is attributable to waste plastic, but ii) that is not based on having a physical component originating from waste plastic.

As used herein, the term “isolated” refers to the characteristic of an object or objects being by itself or themselves and separate from other materials, in motion or static.

As used herein, the terms “mixed plastic waste” and “MPW” refer to a mixture of at least two types of waste plastics including, but not limited to the following plastic types: polyethylene terephthalate (PET), one or more polyolefins (PO), and polyvinylchloride (PVC).

As used herein, the term “molecular reforming” refers to partial oxidation (POX) Gasification and steam reforming.

As used herein, the term “molecular reforming facility” refers to a facility that includes all equipment, lines, and controls necessary to carry out molecular reforming of waste plastic and feedstocks derived therefrom.

As used herein, “non-aqueous” refers to a fluid containing less than five percent of molecular water by weight.

As used herein, the term “overhead” refers to the physical location of a structure that is above a maximum elevation of quantity of particulate plastic solids within an enclosed structure. For example, an “overhead” stream is a stream taken from a vessel at a position that is higher elevation-wise to other streams taken from the vessel, such as a “bottom” stream.

As used herein, the term “partially processed waste plastic” means waste plastic that has been subjected to at least on automated or mechanized sorting, washing, or comminuted step or process. Partially processed waste plastics may originate from, for example, municipal recycling facilities (MRFs) or reclaimers. When partially processed waste plastic is provided to the chemical recycling facility, one or more preprocessing steps may me skipped.

As used herein, the term “physical recycling” (also known as “mechanical recycling”) refers to a waste plastic recycling process that includes a step of melting waste plastic and forming the molten plastic into a new intermediate product (e.g., pellets or sheets) and/or a new end product (e.g., bottles). Generally, physical recycling does not substantially change the chemical structure of the plastic, although some degradation is possible.

As used herein, the term “plastic” may include any organic synthetic polymers that are solid at 25° C. and 1 atmosphere of pressure.

As used herein, the terms “partial oxidation (POX) gasification” or “POX gasification” refers to high temperature conversion of a carbon-containing feed into syngas, (carbon monoxide, hydrogen, and carbon dioxide), where the conversion is carried out in the presence of a less than stoichiometric amount of oxygen. The feed to POX gasification can include solids, liquids, and/or gases.

As used herein, the term “partial oxidation (POX) reaction” refers to all reactions occurring within a partial oxidation (POX) gasifier in the conversion of a carbon-containing feed into syngas, including but not limited to partial oxidation, water gas shift, water gas-primary reactions, Boudouard, oxidation, methanation, hydrogen reforming, steam reforming, and carbon dioxide reforming.

As used herein, the term “predominantly” means more than 50 percent by weight. For example, a predominantly propane stream, composition, feedstock, or product is a stream, composition, feedstock, or product that contains more than 50 weight percent propane.

As used herein, the term “preprocessing” refers to preparing waste plastic for chemical recycling using one or more of the following steps: (i) comminuting, (ii) particulating, (iii) washing, (iv) drying, and/or (v) separating.

As used herein, the term “pyrolysis” refers to thermal decomposition of a feedstock of a biomass and/or a plastic material in solid or liquid form at elevated temperatures in an inert (i.e., substantially molecular oxygen free) atmosphere.

As used herein, the term “pyrolysis char” refers to a carbon-containing composition obtained from pyrolysis that is solid at 200° C. and 1 atm.

As used herein, the terms “pyrolysis gas” and “pygas” refer to a composition obtained from pyrolysis that is gaseous at 25° C. at 1 atm.

As used herein, the term “pyrolysis heavy waxes” refers to C20+ hydrocarbons obtained from pyrolysis that are not pyrolysis char, pyrolysis gas, or pyrolysis oil.

As used herein, the terms “pyrolysis oil” or “pyoil” refers to a composition obtained from pyrolysis that is liquid at 25° C. and 1 atm.

As used herein, the term “pyrolysis residue” refers to a composition obtained from pyrolysis that is not pyrolysis gas or pyrolysis oil and that comprises predominantly pyrolysis char and pyrolysis heavy waxes.

As used herein, the terms “recycle content” and “r-content” refer to being or comprising a composition that is directly and/or indirectly derived from waste plastic.

As used herein, the terms “recycle content CO₂” or “r-CO₂” refer to being or comprising CO₂ that is directly and/or indirectly derived from waste plastic.

As used herein, the terms “recycle content ethylene” or “r-ethylene” refer to being or comprising ethylene that is directly and/or indirectly derived from waste plastic.

As used herein, the terms “recycle content ethylene oxide” or “r-ethylene oxide” refer to being or comprising ethylene oxide that is directly and/or indirectly derived from waste plastic.

As used herein, the terms “recycle content propylene” or “r-propylene” refer to being or comprising propylene that is directly and/or indirectly derived from waste plastic.

As used herein, the terms “recycle content methane” or “r-methane” refer to being or comprising methane that is directly and/or indirectly derived from waste plastic.

As used herein, the terms “r-pyrolysis gas” or “r-pygas” refer to being or comprising a pyrolysis gas that is directly and/or indirectly derived from waste plastic.

As used herein, the terms “r-pyrolysis oil” or “r-pyoil” refer to being or comprising a pyrolysis oil that is directly and/or indirectly derived from waste plastic.

As used herein, the terms “recycle content syngas” or “r-syngas” refer to being or comprising a syngas that is directly and/or indirectly derived from waste plastic.

As used herein, the term “residual” refers to a remaining quantity or amount of an identified product or component that remains from an original source containing the product or component. For example, a “residual pyrolysis oil” may refer to the remaining pyrolysis oil from an initial pyrolysis effluent after the majority of the pyrolysis oil has been previously removed therefrom.

As used herein, the terms “waste plastic” and “plastic waste” refer to used, scrap, and/or discarded plastic materials. The waste plastic fed to the chemical recycling facility may be unprocessed or partially processed.

As used herein, “downstream” means a target unit operation, vessel, or equipment that:
a. is in fluid (liquid or gas) communication, or in piping communication, with an outlet stream from the radiant section of a cracker furnace, optionally through one or more intermediate unit operations, vessels, or equipment, or
b. was in fluid (liquid or gas) communication, or in piping communication, with an outlet stream from the radiant section of a cracker furnace, optionally through one or more intermediate unit operations, vessels, or equipment, provided that the target unit operation, vessel, or equipment remains within the battery limits of the cracker facility (which includes the furnace and all associated downstream separation equipment).

### CLAIMS NOT LIMITED TO DISCLOSED EMBODIMENTS

When a numerical sequence is indicated, it is to be understood that each number is modified the same as the first number or last number in the numerical sequence or in the sentence, e.g., each number is “at least,” or “up to” or “not more than” as the case may be; and each number is in an “or” relationship. For example, “at least 10, 20, 30, 40, 50, 75 wt. % . . . ” means the same as “at least 10 wt. %, or at least 20 wt. %, or at least 30 wt. %, or at least 40 wt. %, or at least 50 wt. %, or at least 75 wt. %,” etc.; and “not more than 90 wt. %, 85, 70, 60 . . . ” means the same as “not more than 90 wt. %, or not more than 85 wt. %, or not more than 70 wt. % . . . ” etc.; and “at least 1%, 2%, 3%, 4%, 5%, 6%, 7%, 8%, 9% or 10% by weight . . . ” means the same as “at least 1 wt. %, or at least 2 wt. %, or at least 3 wt. % . . . ” etc.; and “at least 5, 10, 15, 20 and/or not more than 99, 95, 90 weight percent” means the same as “at least 5 wt. %, or at least 10 wt. %, or at least 15 wt. % or at least 20 wt. % and/or not more than 99 wt. %, or not more than 95 wt. %, or not more than 90 weight percent . . . ” etc.

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A chemical recycling process comprising:
(a) oxidizing at least a portion of a recycle content ethylene in an oxidation reactor to form a recycle content ethylene oxide stream and a recycle content CO₂ stream;
(b) feeding at least a portion of the recycle content CO₂ stream to a POX gasifier; and
(c) gasifying at least a portion of the recycle content CO₂ stream in the POX gasifier to thereby form a recycle content syngas.

2. The process according to claim 1, further comprising melting at least a portion of a solid waste plastic in at least one liquification system to form a liquefied waste plastic, pyrolyzing at least a portion of the liquefied waste plastic in at least one pyrolysis reactor to form a pyrolysis effluent comprising a recycle content pyrolysis gas, a recycle content pyrolysis oil, and a recycle content pyrolysis residue, and cracking at least a portion of the recycle content pyrolysis gas, the recycle content pyrolysis oil, and/or the recycle content pyrolysis residue in at least one cracker furnace to form a cracked product comprising a recycle content methane stream and the recycle content ethylene stream.

3. The process according to claim 2, further comprising bypassing the cracker furnace and introducing at least a portion of the pyrolysis gas directly into a compression system downstream of the cracker furnace.

4. The process according to claim 3, wherein the liquification system, the pyrolysis reactor, and/or the cracker furnace comprise a combustion device comprising one or more burners, further comprising combusting at least a portion of the recycle content methane stream in the combustion device to thereby form a flue gas stream comprising recycle content CO₂.

5. The process according to claim 4, further comprising gasifying at least a portion of the flue gas stream to form additional recycle content syngas.

6. The process according to claim 1, further comprising introducing a molecular oxygen stream into the oxidation reactor and in the presence of a heterogeneous catalyst, and a temperature in the range of 200 to 300° C. or 220 to 280° C.

7. The process according to claim 1, further comprising separating the recycle content ethylene oxide stream and the recycle content CO₂ stream in a first absorber system comprising at least one absorber column and at least one stripper column, and further comprising, prior to the gasifying of step (c), purifying at least a portion of the recycle content CO₂ stream in a second absorber system comprising at least one absorber column and at least one stripper column.

8. The process according to claim 1, wherein the gasifying occurs at:
(i) a temperature of at least 1100° C., and
(ii) a pressure of at least 200 psig.

9. The process according to claim 1, wherein at least a portion of the recycle content ethylene and/or the recycle content methane is produced by a cracking process that includes cracking recycle content pyrolysis oil.

10. The process according to claim 9, wherein at least a portion of the recycle content pyrolysis oil is produced via pyrolysis of waste plastic.

11. The process according to claim 1, wherein at least a portion of the recycle content CO₂ is produced by combusting methane, wherein the methane is produced by cracking recycle content pyrolysis oil.

12. A chemical recycling process comprising:
(a) generating a CO₂ stream by—
i) oxidizing ethylene to form ethylene oxide and CO₂, and
ii) combusting methane to form flue gas comprising CO₂; and
(b) gasifying at least a portion of the CO₂ stream in a POX gasifier to thereby form a syngas.

13. The process according to claim 12, wherein the CO₂ stream is a recycle content CO₂ stream.

14. The process according to claim 12, wherein at least a portion of the ethylene is a recycle content ethylene that is produced by a cracking process that includes cracking recycle content pyrolysis oil.

15. The process according to claim 12, wherein at least a portion of the methane is a recycle content methane that is produced by a cracking process that includes cracking recycle content pyrolysis oil.

16. The process according to claim 12, wherein the CO₂ stream comprises at least 50 mole percent of recycle content CO₂, based on the total molar content of the CO₂ stream.

17. The process according to claim 12, wherein the CO₂ stream comprises at least 0.1 mole percent and not more than 10 mole percent of water, based on the total molar content of the CO₂ stream.

18. The process according to claim 12, wherein at least a portion of the ethylene is produced by a cracking process that includes cracking recycle content naphtha, recycle content diesel, or recycle content ethane or recycle content propane.

19. The process according to claim 12, wherein the POX gasifier comprises a side injector for feeding a gaseous feedstock into the POX gasifier.

20. The process according to claim 12, wherein the POX gasifier does not contain any pyrolysis reaction zones.
